(19) United States
(12) Patent Application Publication
(10) Pub. No.: US 2010/0047264 A1
(43) Pub. Date: Feb. 25, 2010

(54) Immunogenic Affinity-Conjugated Antigen Systems Based on Papaya Mosaic Virus and Uses Thereof

(75) Inventor: Denis Leclerc, Fossambault-sur-le Lac (CA)

Correspondence Address: SWANSON & BRATSCHUN, L.L.C., 8210 SOUTHPARK TERRACE, LITTLETON, CO, 80120, US

(73) Assignee: FOLIA BIOTECH INC., Quebec City, QC (CA)

(21) Appl. No.: 12/514,970

(22) PCT Filed: Oct. 25, 2007
(86) PCT No.: PCT/CA07/01904

§ 371 Date: Jul. 18, 2009

Related U.S. Application Data
(60) Provisional application No. 60/865,997, filed on Nov. 15, 2006.
(60) Provisional application No. 60/886,873, filed on Jan. 26, 2007.

(51) Int. Cl. A61K 39/12; C07K 14/005; C07H 21/04; C12N 7/00

(52) U.S. Cl. 424/185.1; 530/350; 536/23.4; 435/235.1

(57) ABSTRACT

An affinity-conjugated antigen system (ACAS) comprising one or more antigens conjugated via a plurality of affinity moieties to a papaya mosaic virus (PapMV) or virus-like particle (VLP) derived from the coat protein of PapMV is provided. The affinity moieties are molecules or compounds that are capable of specifically binding to the antigen(s) of interest and which can be attached, for example by chemical or genetic means, to the coat protein of the PapMV or PapMV VLP. The ACAS can optionally further comprise one or more additional antigens, which may be the same as, or different to, the conjugated antigen(s) comprised by the ACAS. Also provided are immunogenic compositions, including vaccines, comprising an ACAS. The immunogenic compositions are useful in the treatment, including prevention, of various diseases and disorders for which a humoral and/or cellular response in the animal is required.

### FIELD OF THE INVENTION

The present invention relates to the field of immunogenic compositions, in particular, to immunogenic compositions based on papaya mosaic virus.

### BACKGROUND OF THE INVENTION

Vaccination has provided an effective way for preventing and treating infectious diseases and has led to one of the most significant benefits for public health in the last century. Early vaccination strategies used live, attenuated or inactivated pathogens as the immunogen. Public concern, however, fostered a search for more defined and safer vaccines. This search stimulated a new direction of research, where individual antigens were isolated or recombinantly expressed and injected as immunogens. Such vaccines, however, often required the addition of an adjuvant to generate a sufficient immune response against the antigen, as the isolated protein was typically not sufficiently immunogenic to generate a protective immune response. Although several strong adjuvants were known, such as complete Freund's adjuvant, many had undesirable side effects (Lerner, R. A., et al., Proc. Natl. Acad. Sci. USA 78:3403-3407 (1981); Bhatnagar, P. K., et al., Proc. Natl. Acad. Sci. USA 79:4400-4404 (1982); Neurath, A. R., et al., J. Gen. Virol. 65:1009-1014 (1984); and Muller, G. M., et al., Proc. Natl. Acad. Sci. USA 79:569-573 (1982)).

The use of synthetic peptides as immunogens has also been intensively investigated in an effort to prepare safer and more effective vaccines (Lerner, R. A., Green, N., Alexander, H., Liu, F.-T., Sutcliffe, J. G., and Shinnick, T. M. Proc. Natl. Acad. Sci. USA 78:3403-3407 (1981); Bhatnagar, P. K., Papas, E., Blum, H. E., Milich, D. R., Nitecki, D. Karels, M. J., and Vyas, G. N. Proc. Natl. Acad. Sci. USA 79:4400-4404 (1982); Neurath, A. R., Kent, S. B. H., and Strick, N. J. Gen. Virol. 65:1009-1014 (1984)). The development of synthetic peptide vaccines has, however, also been hampered by the search for desirable adjuvants and carriers.

More recently, research on the principles of discrimination by the immune system between self and foreign has revealed that the degree of organization and the repetitiveness of the antigens on the surfaces of viruses are a very strong signal for an antigen to be recognized as foreign (Bachmann & Zinkernagel, Immunol. Today 17:553 558 (1996)). This property of viral structures was made use of in the design of potential new vaccines based on virus-like particles (VLPs), which combined the immunogenicity of viral structures and the improved safety profile of non-replicable vaccines.

Two VLP vaccines based on animal viruses, hepatitis B virus (HBV) and Human Papilloma Virus (HPV), have been shown to function efficiently in humans (Fagan et al., 1987, J. Med. Virol., 21:49-56; Harper et al., 2004, Lancet, 364:1757-1765). VLPs made from the human papillomavirus (HPV) major capsid protein LI, for example, were shown to provide 100% protection in woman against development of cervical cancers (Ault, K. A., 2006, Obstet. Gynecol. Surv. 61:S26-S31; Harper et al., 2004, Lancet 364:1757-1765, see also International Patent Application PCT/US01/18701 (WO 02/04007)). Platforms such as the bacteriophage Qβ (Maurer et al., 2005, Eur. J. Immunol. 35:2031-2040), the hepatitis B virus VLPs made of the viral core protein (Mihailova et al., 2006, Vaccine 24:4369-4377; Pumpens et al., 2002, Intervirology 45:24-32), and parvovirus VLPs (Antonis et al., 2006, Vaccine 24:5481-5490; Ogasawara et al., 2006, In Vivo 20:319-324) have also shown capacity to carry epitopes and induce a strong antibody response. Similarly, U.S. Pat. No. 6,627,202, describes HBV core proteins comprising antigens crosslinked by HBV capsid-binding peptides for use as epitope delivery systems, including antigens targeted to or derived from various viruses and bacteria.

The use of VLPs from plant viruses as epitope presentation systems has been described. Plant viruses are comprised mainly of proteins that are highly immunogenic, and possess a complex, repetitive and crystalline organisation. In addition, they are phylogenetically distant from the animal immune system, which makes them good candidates for the development of vaccines. For example, cowpea mosaic virus (CPMV), Johnson grass mosaic virus (JGMV), tobacco mosaic virus (TMV), and alfalfa mosaic virus (AIMV) have been modified for the presentation of epitopes of interest (Canizares, M. C. et al., 2005, Immunol. Cell. Biol. 83:263-270; Brennan et al., 2001, Molec. Biol. 17:15-26; Saini and Vrati, 2003, J. Virol. 77:3487-3494). International Patent Application PCT/GB97/01065 (WO 97/39134) describes chimaeric virus-like particles that comprise a coat protein and a non-viral protein, which can be used, for example, for presentation of peptide epitopes. International Patent Application PCT/US01/07355 (WO 01/66778) describes a plant virus coat protein, and specifically a tobacco mosaic virus coat protein, fused via a linker at the N-terminus to a polypeptide of interest, which may include an epitope of a pathogenic microorganism. International Patent Application PCT/US01/20272 (WO 02/00169) describes vaccines comprising either potato virus Y coat protein or a truncated bean yellow mosaic virus coat protein fused to a foreign peptide, and specifically a Newcastle Disease Virus or human immunodeficiency virus (HIV) epitope. Also, U.S. Pat. No. 6,042,832 describes methods of administering fusions of polypeptides, such as pathogen epitopes, with alfalfa mosaic virus or ilarvirus capsid proteins to an animal in order to raise an immune response.

More recently, VLPs derived from the coat protein of papaya mosaic virus (PapMV) and their use as immunopotentiators has been described (International Patent Application No. PCT/CA03/00985 (WO 2004/004761)). Expression of the PapMV coat protein in E. coli leads to the self-assembly of VLPs composed of several hundred CP subunits organised in a repetitive and crystalline manner (Tremblay et al., 2006, FEBS J 273:14). Studies of the expression and purification of PapMV CP deletion constructs further indicate that self-assembly (or multimerization) of the CP subunits is important for function (Lecours et al., 2006, Protein Expression and Purification, 47:273-280). The ability of PapMV VLPs comprising epitopes from either gp100 or the influenza virus M1 protein have been shown to induce MHC class I cross-presentation of the epitopes leading to expansion of specific human T cells (Leclerc, D., et al., J. Virol, 2007, 81(3):1319-26; Epub. ahead of print Nov. 22, 2006). In addition, PapMV VLPs comprising epitopes derived from the hepatitis C virus E2 envelope protein were shown to induce an humoral response in mice toward the PapMV VLP as well as the E2 peptide (Denis et al., 2007, Virology, 363(1): 59-68).

VLPs derived from Potato Virus X (PVX) carrying various antigenic determinants from HIV, HCV, EBV or the influenza virus have been described (European Patent Application No. 1 167 530). The ability of the PVX VLP carrying an HIV epitope to induce antibody production in mice via humoral and cell-mediated pathways is also described. Additional adjuvants were used in conjunction with the PVX VLP to potentiate this effect.

Hepatitis B core protein or parvovirus VLPs have been reported to induce a CTL response even when they do not carry genetic information (Ruedl et al., 2002, Eur. J. Immunol. 32; 818-825; Martinez et al., 2003, Virology, 305; 428-435) and can not actively replicate in the cells where they are invaginated. The cross-presentation of such VLPs carrying an epitope from lymophocytic choriomeningitis virus (LCMV) or chicken egg albumin by dendritic cells in vivo has also been described (Ruedl et al., 2002, ibid.; Morón, et al., 2003, J. Immunol. 171:2242-2250). The ability of a hepatitis B core protein VLP carrying an epitope from LCMV to prime a CTL response has also been described, however, this VLP was unable to induce the CTL response when administered alone and failed to mediate effective protection from viral challenge. An effective CTL response was induced only when the VLP was used in conjunction with anti-CD40 antibodies or CpG oligonucleotides (Storni, et al., 2002, J. Immunol. 168:2880-2886). An earlier report indicated that porcine parvovirus-like particles (PPMV) carrying a peptide from LCMV were able to protect mice against a lethal LCMV challenge (Sedlik, et al., 2000, J. Virol. 74:5769-5775).

Papaya mosaic virus VLPs fused to affinity peptides have been proposed as an alternative to monoclonal antibodies in the detection of fungal diseases (Morin et al., 2007, J. Biotechnology, 128: 423-434 [epub ahead of print Oct. 26, 2006]). VLPs were developed that were capable of binding Plasmodiophora brassicae spores with high avidity and binding of one construct to the spores was demonstrated to be at a level comparable to that of polyclonal antibodies.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide immunogenic affinity-conjugated antigen systems based on papaya mosaic virus and uses thereof. In accordance with one aspect of the present invention there is provided an affinity-conjugated antigen system comprising one or more antigens and a papaya mosaic virus (PapMV) or a virus-like particle (VLP) derived from PapMV coat protein, said PapMV or VLP comprising a plurality of affinity moieties attached to coat proteins of the PapMV or VLP, said affinity moieties capable of binding said one or more antigens, wherein said system is capable of inducing an immune response in an animal.

In accordance with another aspect of the present invention, there is provided an immunogenic composition comprising an affinity-conjugated antigen system of the invention and a pharmaceutically acceptable carrier.

In accordance with another aspect of the present invention, there is provided a method of inducing an immune response in an animal comprising administering to said animal an effective amount of an affinity-conjugated antigen system of the invention.

In accordance with another aspect of the present invention, there is provided a method of preventing or treating a disease or disorder in an animal, said method comprising administering to said animal an effective amount of an antigen presenting system of the invention.

In accordance with another aspect of the present invention, there is provided a use of an effective amount of an affinity-conjugated antigen system of the invention to induce an immune response in an animal in need thereof.

In accordance with another aspect of the present invention, there is provided a use of an effective amount of an affinity-conjugated antigen system of the invention to prevent or treat a disease or disorder in an animal in need thereof.

In accordance with another aspect of the present invention, there is provided a use of a papaya mosaic virus (PapMV) or a virus-like particle (VLP) derived from PapMV coat protein in the manufacture of a medicament, said PapMV or VLP comprising a plurality of affinity moieties attached to coat proteins of said PapMV or VLP.

In accordance with another aspect of the present invention, there is provided a use of an affinity-conjugated antigen system of the invention in the manufacture of a medicament.

In accordance with another aspect of the present invention, there is provided a method of preparing an immunogenic composition comprising admixing one or more antigens with a papaya mosaic virus (PapMV) or a virus-like particle (VLP) derived from PapMV coat protein, said PapMV or VLP comprising a plurality of affinity moieties attached to coat proteins of said PapMV or VLP, said affinity moieties capable of binding said one or more antigens.

In accordance with another aspect of the present invention, there is provided an immunogenic composition prepared by a method comprising admixing one or more antigens with a papaya mosaic virus (PapMV) or a virus-like particle (VLP) derived from PapMV coat protein, said PapMV or VLP comprising a plurality of affinity moieties attached to coat proteins of said PapMV or VLP, said affinity moieties capable of binding said one or more antigens.

In accordance with another aspect of the present invention, there is provided a fusion protein comprising a papaya mosaic virus (PapMV) coat protein fused to an affinity peptide capable of binding to HCV core protein.

In accordance with another aspect of the present invention, there is provided an isolated polynucleotide encoding a fusion protein comprising a papaya mosaic virus (PapMV) coat protein fused to an affinity peptide capable of binding to HCV core protein.

In accordance with another aspect of the present invention, there is provided a use of a fusion protein comprising a papaya mosaic virus (PapMV) coat protein fused to an affinity peptide capable of binding to HCV core protein, or a polynucleotide encoding a fusion protein comprising a papaya mosaic virus (PapMV) coat protein fused to an affinity peptide capable of binding to HCV core protein to prepare a virus-like particle.

### BRIEF DESCRIPTION OF THE DRAWINGS

These and other features of the invention will become more apparent in the following detailed description in which reference is made to the appended drawings.

FIG. 1 presents (A) the amino acid sequence for the papaya mosaic virus coat (or capsid) protein (GenBank Accession No. NP_{—}044334.1; SEQ ID NO:1), (B) the nucleotide sequence encoding the papaya mosaic coat protein (GenBank Accession No. NC_{—}001748 (nucleotides 5889-6536); SEQ ID NO:2), and (C) the amino acid sequence of the mutant PapMV coat protein CPΔN5 (SEQ ID NO:3).

FIG. 2 illustrates the ability of PapMV to strengthen antibody responses to the model antigens (A) hen egg lysozyme (HEL) and (B) ovalbumin (OVA) in BALB/c mice (three per group) immunized on day 0 with antigen alone, antigen plus PapMV, Freund's complete adjuvant (FCA) or LPS from E. coli O111:B4. A representative result from 2 experiments is shown. The antibodies of the serum collected from the immunized animals were isotyped by ELISA on the model antigens (HEL or OVA) for (C) IgG1, (D) IgG 2a and (E) IgG2b.

FIG. 3 presents (A) a schematic representation of the recombinant constructs, which comprise a fusion at the C-terminus of the PapMV coat protein of the affinity peptide to OmpC or to OmpF (constructs PapMV OmpC and PapMV OmpF, respectively); (B) SDS-PAGE showing the profile of the purified proteins PapMV, PapMV OmpC, PapMV OmpF, OmpC and OmpF, [First lane: molecular weight markers, second lane; PapMV VLPs, third lane; PapMV OmpC VLPs, fourth lane; PapMV OmpF VLPs, fifth lane; purified OmpC, sixth lane; purified OmpF], and (C) an electron micrograph of the high-speed pellet of the recombinant PapMV OmpC and PapMV OmpF VLPs.

FIG. 4 illustrates high avidity binding of the PapMV VLPs to their respective antigen; (A) presents an ELISA showing the binding the high avidity PapMV OmpC VLPs to the OmpC antigen; (B) presents an ELISA showing the binding the high avidity PapMV OmpF VLPs to the OmpF antigen.

FIG. 5 presents the results of a protection assay against S. typhi challenge in mice, (A) depicts the protective capacity against 100 LD₅₀ of S. typhi in mice immunised with OmpC alone and mice immunised with a preparation containing OmpC+PapMV OmpC VLPs; (B) depicts the protective capacity against 100 LD₅₀ of S. typhi in mice immunised with OmpF alone and mice immunised with a preparation containing OmpF+PapMV OmpF VLPs; (C) depicts the protective capacity against 500 LD₅₀ of S. typhi in mice immunised with OmpC alone and mice immunised with a preparation containing OmpC+PapMV OmpC VLPs, and (D) depicts the protective capacity against 500 LD₅₀ of S. typhi in mice immunised with OmpF alone and mice immunised with a preparation containing OmpF+PapMV OmpF VLPs.

FIG. 6 illustrates the evaluation of the antibody response directed to OmpC; the IgG response to OmpC of the isotypes IgG1, IgG2a, IgG2b and IgG3 was measured between mice immunised with OmpC or a vaccine comprising OmpC+PapMV OmpC VLPs.

FIG. 7 illustrates that co-immunization of OmpC and PapMV OmpC to mice followed by challenge with S. typhi favours the long lasting active protection of mice against S. typhi infection (as illustrated by % survival) when compared to immunization with OmpC or PapMV OmpC alone.

FIG. 8 illustrates that PapMV virus increased the protective capacity of OmpC porin; the results of a challenge performed with 100 (filled symbols) or 500 lethal dose 50 (LD50) (open symbols) of S. typhi, with survival rate recorded for 10 days after the challenge, are shown.

FIG. 9 presents the amino acid sequence (SEQ ID NO:4) of the OmpC precursor protein from Salmonella enterica subsp. enterica serovar Typhi Ty2 (GenBank Accession No. P0A264);

FIG. 10 presents the amino acid sequence (SEQ ID NO:5) of the OmpF precursor protein from Salmonella enterica subsp. enterica serovar Typhi CT18 (GenBank Accession No. CAD05399).

FIG. 11 presents the amino acid sequence of (A) PapMV coat protein comprising an affinity peptide for binding to OmpC [SEQ ID NO:6], and (B) PapMV coat protein comprising an affinity peptide for binding to OmpF [SEQ ID NO:7]. Differences between the cloned and wild-type sequence are marked in bold and underlined; the affinity peptide sequence is underlined, and the histidine tag is shown in italics.

FIG. 12 presents (A) an electron micrograph of PapMV VLPs purified from E. coli; (B) an electron micrograph of PapMV VLPs comprising the affinity peptide STASYTR [SEQ ID NO:8] (PapMVCP-STASYTR); (C) an ELISA showing the binding of PapMVCP-STASYTR to HCV core protein (1-170) NLPs (1 μg/ml). The grey bars represent the signal obtained with PapMVCP-STASYTR; the dotted bars represent the background signal obtained with PapMV VLPs alone; and (D) as C except that 1 μg/ml of the free HCV core protein (1-170) was used to load the ELISA plate. All ELISAs were revealed with a polyclonal rabbit antibody directed to the PapMV coat protein and a goat anti-rabbit antibody conjugated to alkaline phosphatase.

FIG. 13 illustrates the immune response in mice against HCV-Core protein and PapMV VLPs: (A) presents antibody titers (total IgG) against HCV core protein (1-82) NLPs (“core”), PapMV VLPs comprising the affinity peptide STASYTR [SEQ ID NO:8] (“PapSTA”), and PapSTA in combination with HCV core protein (1-82) NLPs (“PapSTA+core”); and (B) titers of recombinant vaccinia virus recovered from both ovaries of mice vaccinated with PapSTA, HCV core protein (1-82) NLPs, or PapSTA in combination with HCV core protein (1-82) NLPs, and subsequently challenged with recombinant vaccinia virus expressing amino acids 1-382 of the HCV polyprotein.

FIG. 14 presents (A) the nucleotide sequence of the PapMV coat protein comprising an affinity peptide for binding to HCV core protein fragments (PapMVCP-STASYTR; SEQ ID NO:48), and (B) the amino acid sequence of the PapMVCP-STASYTR construct (SEQ ID NO: 42). The start codon is in bold italics, the stop codon is in bold and underlined, and the histidine tag is shown in italics.

### DETAILED DESCRIPTION OF THE INVENTION

An affinity-conjugated antigen system (ACAS) comprising one or more antigens conjugated via a plurality of affinity moieties to a papaya mosaic virus (PapMV) or a virus-like particle (VLP) derived from the coat protein of PapMV is provided. By “derived from” it is meant that the VLP comprises coat proteins that have an amino acid sequence substantially identical to the sequence of the wild-type coat protein. The affinity moieties are molecules or compounds that are capable of specifically binding to the antigen(s) of interest and which can be attached, for example by chemical or genetic means, to the coat protein of the PapMV or PapMV VLP to form an affinity PapMV (“aPapMV”) or affinity VLP (“aVLP”). In one embodiment, the aPapMV and aVLPs according to the present invention are particularly useful for conjugating large antigens, such as macromolecules.

While the antigens are described herein as being “conjugated” to the aPapMV or aVLP in the ACAS of the present invention, it is contemplated that, depending on the local environment, the antigen(s) and aPapMV/aVLP in the ACAS may be present in non-conjugated form some, or all, of the time. Similarly, it is contemplated that not all of antigen present in the ACAS may be conjugated to its cognate aPapMV/aVLP. For example, as would be appreciated by the skilled worker, conditions of very high or low pH or salt concentrations, or high dilution of the ACAS, could affect the binding of the antigen(s) to their cognate aPapMV/aVLP.

The ACAS of the present invention can optionally further comprise one or more additional isolated antigens (or AlAs). In the context of the present invention, an AIA is an antigen other than the antigen(s) that the affinity moieties comprised by the aPapMV/aVLP are capable of binding.

In accordance with one aspect of the present invention, the ACAS is immunogenic and capable of inducing an immune response when administered to an animal. The immune response may be a humoral response, a cellular response or both. The present invention thus provides for immunogenic compositions, including vaccines, comprising an ACAS. The immunogenic compositions are useful in the treatment, including prevention, of various diseases and disorders for which a humoral and/or cellular response in the animal is required. In one embodiment of the present invention, the ACAS is particularly useful in the treatment, including prevention, of diseases or disorders which require participation of the humoral immune response of an animal.

### DEFINITIONS

Unless defined otherwise, all technical and scientific terms used herein have the same meaning as commonly understood by one of ordinary skill in the art to which this invention belongs.

As used herein, the term “about” refers to approximately a +/−10% variation from a given value. It is to be understood that such a variation is always included in any given value provided herein, whether or not it is specifically referred to.

The term “adjuvant,” as used herein, refers to an agent that augments, stimulates, actuates, potentiates and/or modulates an immune response in an animal.

The term “immunogenic,” as used herein, refers to the ability of a substance to induce a detectable immune response in an animal.

The terms “immune stimulation” and “immunostimulation” as used interchangeably herein, refer to the ability of a molecule, such as a PapMV or PapMV VLP, that is unrelated to an animal pathogen or disease to provide protection to against infection by the pathogen or against the disease by stimulating the immune system and/or improving the capacity of the immune system to respond to the infection or disease. Immunostimulation may have a prophylactic effect, a therapeutic effect, or a combination thereof.

The term “immune response,” as used herein, refers to an alteration in the reactivity of the immune system of an animal in response to administration of a substance (for example, a compound, molecule, material or the like) and may involve antibody production, induction of cell-mediated immunity, complement activation, development of immunological tolerance, or a combination thereof.

The term “vaccination,” as used herein, refers to the administration of a vaccine to a subject for the purposes of generating an immunoprotective response. Vaccination may have a prophylactic effect, a therapeutic effect, or a combination thereof. Vaccination can be accomplished using various methods depending on the subject to be treated including, but not limited to, parenteral administration, such as intraperitoneal injection (i.p.), intravenous injection (i.v.) or intramuscular injection (i.m.); oral administration; intranasal administration; intradermal administration; transdermal administration and immersion.

The term “vaccine,” as used herein, refers to a composition capable of producing an immunoprotective response.

The term “disease or disorder causing agent,” as used herein, refers to an agent that is capable of causing a disease or disorder in a host. Non-limiting examples include agents which cause cancers, infectious diseases, allergic reactions, autoimmune diseases, or can induce an immune response against drugs, hormones or toxins. Infectious diseases include those caused by pathogens, such as, for example, species of bacteria, viruses, protozoa, fungi and parasites.

The term “pathogen,” as used herein, refers to an organism capable of causing a disease or disorder in a host including, but not limited to, bacteria, viruses, protozoa, fungi and parasites.

“Naturally-occurring,” as used herein, as applied to an object, refers to the fact that the object can be found in nature. For example, an organism (including a virus), or a polypeptide or polynucleotide sequence that is present in an organism that can be isolated from a source in nature and which has not been intentionally modified by man in the laboratory is naturally-occurring.

The terms “polypeptide” or “peptide” as used herein is intended to mean a molecule in which there is at least four amino acids linked by peptide bonds.

The expression “viral nucleic acid,” as used herein, may be the genome (or a majority thereof) of a virus, or a nucleic acid molecule complementary in base sequence to that genome. A DNA molecule that is complementary to viral RNA is also considered viral nucleic acid, as is a RNA molecule that is complementary in base sequence to viral DNA.

The term “virus-like particle” (VLP), as used herein, refers to a self-assembling particle which has a similar physical appearance to a virus particle. The VLP may or may not comprise viral nucleic acids. VLPs are generally incapable of replication.

The term “pseudovirus,” as used herein, refers to a VLP that comprises nucleic acid sequences, such as DNA or RNA, including nucleic acids in plasmid form. Pseudoviruses are generally incapable of replication.

The terms “immunogen” and “antigen” as used herein refer to a molecule, molecules, a portion or portions of a molecule, or a combination of molecules, up to and including whole cells and tissues, which are capable of inducing an immune response in a subject alone or in combination with an adjuvant. The immunogen/antigen may comprise a single epitope or may comprise a plurality of epitopes. The term thus encompasses peptides, carbohydrates, proteins, nucleic acids, and various microorganisms, in whole or in part, including viruses, bacteria and parasites. Haptens are also considered to be encompassed by the terms “immunogen” and “antigen” as used herein.

The term “prime” and grammatical variations thereof, as used herein, means to stimulate and/or actuate an immune response against an antigen in an animal prior to administering a booster vaccination with the antigen.

As used herein, the terms “treat,” “treated,” or “treating” when used with respect to a disease or pathogen refers to a treatment which increases the resistance of a subject to the disease or to infection with a pathogen (i.e. decreases the likelihood that the subject will contract the disease or become infected with the pathogen) as well as a treatment after the subject has contracted the disease or become infected in order to fight a disease or infection (for example, reduce, eliminate, ameliorate or stabilise a disease or infection).

The term “subject” or “patient” as used herein refers to an animal in need of treatment.

The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep or other hoofed animals, dogs, cats, chickens, ducks, non-human primates, guinea pigs, rabbits, ferrets, rats, hamsters and mice.

The term “substantially identical,” as used herein in relation to a nucleic acid or amino acid sequence indicates that, when optimally aligned, for example using the methods described below, the nucleic acid or amino acid sequence shares at least 70%, at least 75%, at least 80%, at least 85%, at least 90%, at least 95%, at least 96%, at least 97%, at least 98% or at least 99% sequence identity with a defined second nucleic acid or amino acid sequence (or “reference sequence”). “Substantial identity” may be used to refer to various types and lengths of sequence, such as full-length sequence, functional domains, coding and/or regulatory sequences, promoters, and genomic sequences. Percent identity between two amino acid or nucleic acid sequences can be determined in various ways that are within the skill of a worker in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. For nucleic acids, the length of comparison sequences will generally be at least 25 nucleotides, but may be at least 50, at least 100, at least 125, at least 150, at least 200, at least 250, at least 300, at least 350, at least 400, at least 450, at least 500, at least 550, or at least 600 nucleotides, or it may be the full-length of the nucleic acid sequence.

The terms “corresponding to” or “corresponds to” indicate that a nucleic acid sequence is identical to all or a portion of a reference nucleic acid sequence. In contradistinction, the term “complementary to” is used herein to indicate that the nucleic acid sequence is identical to all or a portion of the complementary strand of a reference nucleic acid sequence. For illustration, the nucleic acid sequence “TATAC” corresponds to a reference sequence “TATAC” and is complementary to a reference sequence “GTATA.”

### Affinity-Conjugated Antigen System (ACAS)

The affinity-conjugated antigen system (ACAS) of the present invention comprises a PapMV or VLP, which has been modified to comprise a plurality of affinity moieties that are capable of binding the antigen(s) of interest (referred to as an affinity PapMV (aPapMV) or an affinity VLP (aVLP), respectively), together with the one or more antigens of interest which are conjugated to the aPapMV or aVLP via the affinity moieties. The ACAS may optionally comprise one or more additional isolated antigens (or AlAs), as noted above.

Affinity Papaya Mosaic Virus (aPapMV) and Affinity PapMV VLPs (aVLPs)

An aPapMV suitable for inclusion in the ACAS of the present invention is a PapMV the coat protein of which has been modified, for example chemically, to comprise a plurality of affinity moieties. The PapMV used to prepare the aPapMV can be a wild-type PapMV or a naturally occurring variant thereof.

The PapMV aVLPs suitable for inclusion in the ACAS are formed from recombinant PapMV coat proteins that can multimerize and self-assemble to form a VLP. When assembled, each VLP comprises a long helical array of coat protein subunits. The wild-type virus comprises over 1200 coat protein subunits and is about 500 nm in length. PapMV VLPs that are either shorter or longer than the wild-type virus can still, however, be effective. In one embodiment of the present invention, the VLP comprises at least 40 coat protein subunits. In another embodiment, the VLP comprises between about 40 and about 1600 coat protein subunits. In an alternative embodiment, the VLP is at least 40 nm in length. In another embodiment, the VLP is between about 40 nm and about 600 nm in length.

The aVLPs can be prepared from a plurality of recombinant coat proteins having identical amino acid sequences, such that the final aVLP when assembled comprises identical coat protein subunits, or the aVLP can be prepared from a plurality of recombinant coat proteins having different amino acid sequences, such that the final aVLP when assembled comprises variations in its coat protein subunits.

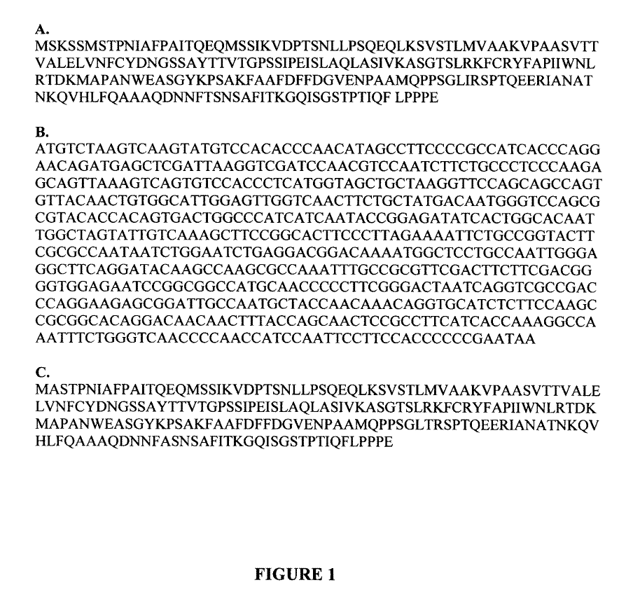

The coat protein used to form the aVLP can be the entire PapMV coat protein, or part thereof, or it can be a genetically modified version of the PapMV coat protein, for example, comprising one or more amino acid deletions, insertions, replacements and the like, provided that the coat protein retains the ability to multimerize and assemble into a VLP. The amino acid sequence of the wild-type PapMV coat (or capsid) protein is known in the art (see, Sit et al., 1989, J. Gen. Virol., 70:2325-2331, and GenBank Accession No. NP_{—}044334.1) and is provided herein as SEQ ID NO:1 (see FIG. 1A). The nucleotide sequence of the PapMV coat protein is also known in the art (see, Sit, et al., ibid., and GenBank Accession No. NC_{—}001748 (nucleotides 5889-6536)) and is provided herein as SEQ ID NO:2 (see FIG. 1B).

As noted above, the amino acid sequence of the recombinant PapMV coat protein comprised by the aVLP need not correspond precisely to the parental sequence, i.e. it may be a modified or “variant sequence.” For example, the recombinant protein may be mutagenized by substitution, insertion or deletion of one or more amino acid residues so that the residue at that site does not correspond to the parental (reference) sequence. One skilled in the art will appreciate, however, that such mutations will not be extensive and will not dramatically affect the ability of the recombinant coat protein to multimerize and assemble into a VLP. The ability of a variant version of the PapMV coat protein to assemble into multimers and form VLPs can be assessed, for example, by electron microscopy following standard techniques, such as the exemplary methods set out in the Examples provided herein.

Recombinant coat proteins that are fragments of the wild-type protein that retain the ability to multimerize and assemble into a VLP (i.e. are “functional” fragments) are, therefore, also contemplated by the present invention. For example, a fragment may comprise a deletion of one or more amino acids from the N-terminus, the C-terminus, or the interior of the protein, or a combination thereof. In general, functional fragments are at least 100 amino acids in length. In one embodiment of the present invention, functional fragments are at least 150 amino acids, at least 160 amino acids, at least 170 amino acids, at least 180 amino acids, and at least 190 amino acids in length. Deletions made at the N-terminus of the protein should generally delete fewer than 25 amino acids in order to retain the ability of the protein to multimerize.

In accordance with the present invention, when a recombinant coat protein comprises a variant sequence, the variant sequence is at least about 70% identical to the reference sequence. In one embodiment, the variant sequence is at least about 75% identical to the reference sequence. In other embodiments, the variant sequence is at least about 80%, at least about 85%, at least about 90%, at least about 95%, and at least about 97% identical to the reference sequence. In a specific embodiment, the reference amino acid sequence is SEQ ID NO:1.

In one embodiment of the present invention, the aVLP comprises a genetically modified (i.e. variant) version of the PapMV coat protein. In another embodiment, the PapMV coat protein has been genetically modified to delete amino acids from the N- or C-terminus of the protein and/or to include one or more amino acid substitutions. In a further embodiment, the PapMV coat protein has been genetically modified to delete between about 1 and about 10 amino acids from the N- or C-terminus of the protein.

In a specific embodiment, the PapMV coat protein has been genetically modified to remove one of the two methionine codons that occur proximal to the N-terminus of the protein (i.e. at positions 1 and 6 of SEQ ID NO:1) and can initiate translation. Removal of one of the translation initiation codons allows a homogeneous population of proteins to be produced. The selected methionine codon can be removed, for example, by substituting one or more of the nucleotides that make up the codon such that the codon codes for an amino acid other than methionine, or becomes a nonsense codon. Alternatively all or part of the codon, or the 5′ region of the nucleic acid encoding the protein that includes the selected codon, can be deleted. In a specific embodiment of the present invention, the PapMV coat protein has been genetically modified to delete between 1 and 5 amino acids from the N-terminus of the protein. In a further embodiment, the genetically modified PapMV coat protein has an amino acid sequence substantially identical to SEQ ID NO:3.

When the recombinant coat protein comprises a variant sequence that contains one or more amino acid substitutions, these can be “conservative” substitutions or “non-conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group. A non-conservative substitution involves the replacement of one amino acid residue by another residue having different side chain properties, for example, replacement of an acidic residue with a neutral or basic residue, replacement of a neutral residue with an acidic or basic residue, replacement of a hydrophobic residue with a hydrophilic residue, and the like.

In one embodiment of the present invention, the variant sequence comprises one or more non-conservative substitutions. Replacement of one amino acid with another having different properties may improve the properties of the coat protein. For example, as described herein, mutation of residue 128 of the coat protein improves assembly of the protein into VLPs. In one embodiment of the present invention, therefore, the coat protein comprises a mutation at residue 128 of the coat protein in which the glutamic residue at this position is substituted with a neutral residue. In a further embodiment, the glutamic residue at position 128 is substituted with an alanine residue.

Likewise, the nucleic acid sequence encoding the recombinant coat protein need not correspond precisely to the parental reference sequence but may vary by virtue of the degeneracy of the genetic code and/or such that it encodes a variant amino acid sequence as described above. In one embodiment of the present invention, therefore, the nucleic acid sequence encoding the recombinant coat protein is at least about 70% identical to the reference sequence. In another embodiment, the nucleic acid sequence encoding the recombinant coat protein is at least about 75% identical to the reference sequence. In other embodiments, the nucleic acid sequence encoding the recombinant coat protein is at least about 80%, at least about 85% or at least about 90% identical to the reference sequence. In a specific embodiment, the reference nucleic acid sequence is SEQ ID NO:2.

As described in more detail below, the coat protein comprised by the aVLP is also modified, for example, chemically or genetically, to include one or more affinity moieties for conjugation with the one or more antigens comprised by the ACAS. In one embodiment, the aVLP comprises a coat protein genetically fused to one or more affinity proteins or peptides.

### Affinity Moieties

The affinity moieties selected for use in the ACAS of the present invention are preferably capable of specifically binding the antigen of interest and of being attached, for example by chemical or genetic means, to a PapMV coat protein. Various affinity moieties are known in the art and suitable affinity moieties for binding a target antigen of interest can be readily selected by a worker skilled in the art.

Examples of suitable affinity moieties include, but are not limited to, antibodies and antibody fragments (such as Fab fragments, Fab′ fragments, Fab′-SH, fragments F(ab′)₂ fragments, Fv fragments, diabodies, and single-chain Fv (scFv) molecules), streptavidin (to bind biotin labelled antigens), natural ligands (or the binding domains of ligands), peptides or protein fragments (such as receptor protein fragments) that specifically bind the antigen. Synthetic affinity moieties having specificity for an antigen of the invention are also herein contemplated.

Examples of ligands include, but are not limited to, proteins, modified proteins (for example, glycoproteins), carbohydrates, proteoglycans, lipids, mucin molecules, and other similar molecules known in the art.

Various affinity moieties capable of binding a given antigen are known in the art and numerous antibodies, antibody fragments, receptors and receptor fragments, and ligands are commercially available (for example, from Invitrogen Corp., Carlsbad, Calif.; Santa Cruz Biotechnology, Santa Cruz, Calif.; ABR-Affinity Bioreagents, Golden, Colo., and Abcam Inc., Cambridge, Mass., amongst others). In addition, methods of producing antibodies and antibody fragments specific for a given target molecule are known in the art (see, for example, Current Protocols in Immunology, ed. Coligan et al., J. Wiley & Sons, New York, N.Y.).

With respect to ligands, a web-based public accessible database for Protein-Ligand INTeractions (ProLINT), includes binding data, sequence and structural information regarding proteins, structural information regarding ligands, and experimental details regarding protein-ligand interactions. Knowledge about the interactions between ligands and their target proteins can be characterized using QSAR Analysis (Kitajima et al., 2002. Genomic Information, 13:498-499) and used in the design of novel ligands using techniques known in the art.

Suitable peptides or antibodies (including antibody fragments) for use as affinity moieties can also be selected by art-known techniques, such as phage or yeast display techniques. The peptides or antibodies can be naturally occurring, recombinant, synthetic, or a combination of these. For example, the peptide can be a fragment of a naturally occurring protein or polypeptide. The term peptide as used herein also encompasses peptide analogues, peptide derivatives and peptidomimetic compounds. Such compounds are well known in the art and may have advantages over naturally occurring peptides, including, for example, greater chemical stability, increased resistance to proteolytic degradation, enhanced pharmacological properties (such as, half-life, absorption, potency and efficacy) and/or reduced antigenicity.

In one embodiment of the present invention, the affinity moiety is a peptide. Suitable peptides can range from about 3 amino acids in length to about 50 amino acids in length. In accordance with one embodiment of the invention, an affinity peptide suitable for use in the ACAS is at least 5 amino acids in length. In accordance with another embodiment of the invention, an affinity peptide suitable for use in the ACAS is at least 7 amino acids in length. In accordance with another embodiment of the invention, an affinity peptide suitable for use in the ACAS is between about 5 and about 50 amino acids in length. In accordance with another embodiment of the invention, an affinity peptide suitable for use in the ACAS is between about 7 and about 50 amino acids in length. In other embodiments of the present invention, an affinity peptide suitable for use in the ACAS between about 5 and about 45 amino acids in length, between about 5 and about 40 amino acids in length, between about 5 and about 35 amino acids in length and between about 5 and about 30 amino acids in length. In accordance with a specific embodiment of the invention, an affinity peptide suitable for use in the ACAS is 3, 4, 5, 6, 7, 8, 9, 10, 11, 12, 13, 14 or 15 amino acids in length. As would be understood by a worker skilled in the art, when the peptide is to be genetically fused to the PapMV coat protein, the length of the peptide selected should not interfere with the ability of the coat protein to self-assemble into VLPs.

When the affinity moieties comprised by the PapMV or VLP comprise a peptide, the affinity moiety can be a single peptide or it can comprise a tandem or multiple arrangement of peptides.

A spacer can be included between the affinity moiety and the coat protein if desired in order to facilitate the binding of large antigens. Suitable spacers include short stretches of neutral amino acids, such as glycine. For example, a stretch of between about 3 and about 10 neutral amino acids. In one embodiment, a stretch of between about 3 and about 10 amino acids is inserted between the PapMV coat protein and the affinity moiety.

As noted above, phage display can be used to select specific peptides that bind to an antigenic protein of interest using standard techniques (see, for example, Current Protocols in Immunology, ed. Coligan et al., J. Wiley & Sons, New York, N.Y.) and/or commercially available phage display kits (for example, the Ph.D. series of kits available from New England Biolabs, and the T7-Select® kit available from Novagen). An example of selection of peptides by phage display is also provided in Examples 3 and 7, below.

Representative peptides that bind a given antigen that were identified by phage display are shown in Table 1. One skilled in the art will appreciate that these peptides are examples only and that other peptides having an affinity for an antigen of interest can be readily identified using art-known techniques. Truncated versions, for example comprising at least 4 consecutive amino acids, of the sequences set forth in Table 1 are also contemplated. In accordance with one embodiment of the present invention, there is provided an ACAS comprising a PapMV or VLP that includes one or more affinity peptides comprising all or a part of the sequence set forth in SEQ ID NO:9, SEQ ID NO:10, SEQ ID NO:11, SEQ ID NO:12, SEQ ID NO:13 or SEQ ID NO:14. In another embodiment, there is provided an ACAS comprising a PapMV or VLP that includes one or more affinity peptides comprising all or a part of the sequence as set forth in SEQ ID NO:8, SEQ ID NO:15, SEQ ID NO:16, SEQ ID NO:17, SEQ ID NO:18, SEQ ID NO:19 or SEQ ID NO:20.

TABLE 1
Examples of affinity peptides selected by
phage display

| | | SEQ |
|---|---|---|
| | Selected | ID |
| Target | affinity peptides | NO |
| S. typhi OmpC | SLSLIQT | 9 |
| S. typhi OmpC | EAKGLIR | 10 |
| S. lyphi OmpC | TATYLLD | 11 |
| S. typhi OmpF | FHENWPS | 12 |
| S. typhi OmpF | FHEFWPT | 13 |
| S. typhi OmpF | FHEXWPT, where X is N or F | 14 |
| HCV Core | STASYIR | 8 |
| HCV Core | NASSLRS | 15 |
| HCV Core | HSPKNLH | 16 |
| HCV Core | NTPQGMT | 17 |
| HCV Core | GPSTPIR | 18 |
| HCV Core | GVQIMGR | 19 |
| HCV Core | SIQYTGV | 20 |

### Antigens

The affinity-conjugated antigen system (ACAS) of the present invention comprises one or more antigens conjugated to the aPapMV or aVLP via its affinity moieties. The ACAS may also optionally comprise one or more AlAs, which may be the same as, or different to, the conjugated antigen(s).

A wide variety of antigens associated with various diseases or disorders are known in the art. Appropriate antigens for inclusion in the ACAS of the invention can be readily selected by one skilled in the art based on, for example, the desired end use of the composition such as the disease or disorder against which it is to be directed and/or the animal to which it is to be administered.

For example, the antigen can be derived from an agent capable of causing a disease or disorder in an animal, such as a cancer, infectious disease, allergic reaction, or autoimmune disease, or it can be an antigen suitable for use to induce an immune response against drugs, hormones or a toxin-associated disease or disorder. The antigen may be derived from a pathogen known in the art, such as, for example, a bacterium, virus, protozoan, fungus, parasite, or infectious particle, such as a prion, or it may be a tumour-associated antigen, a self-antigen or an allergen.

The antigen(s) for incorporation into the ACAS can vary in size and may be, for example, peptides, proteins, nucleic acids, polysaccharides, small molecules, or a combination thereof up to and including a whole pathogen or a portion thereof, for example, a live, inactivated or attenuated version of a pathogen. In one embodiment, the antigen(s) incorporated into the ACAS are macromolecules, for example, proteins (including glycoproteins, lipoproteins and the like), large fragments of proteins (for example, about 20 amino acids or greater in length), polysaccharides, polysaccharide fragments, nucleic acids, nucleic acid fragments, whole pathogens or portions of pathogens. In another embodiment, the antigen(s) incorporated into the ACAS are short fragments of proteins or peptides (for example, between about 4 and about 20 amino acids in length).

When the ACAS is to comprise more than one antigen, the antigens selected for inclusion in the ACAS can be the same, or they can be different, and may be derived from a single source or from a plurality of sources. The antigens can each have a single epitope capable of triggering a specific immune response, or each antigen may comprise more than one epitope.

The antigen may comprise epitopes recognised by surface structures on T cells, B cells, NK cells, macrophages, Class I or Class II APC associated cell surface structures, or a combination thereof. In one embodiment, the present invention contemplates that the ACAS is especially useful for weakly immunogenic antigens.

In addition to known antigens, antigens for inclusion in the ACAS of the invention may also be selected from pathogens or other sources of interest by art known methods and screened for their ability to induce an immune response in an animal using standard immunological techniques known in the art. For example, methods for prediction of epitopes within an antigenic protein are described in Nussinov R and Wolfson H J, Comb Chem High Throughput Screen (1999) 2(5):261, and methods of predicting CTL epitopes are described in Rothbard et al., EMBO J. (1988) 7:93-100 and in de Groot M S et al., Vaccine (2001) 19(31):4385-95. Other methods are described in Rammensee H-G. et al., Immunogenetics (1995) 41:178-228 and Schirle M et al., Eur J Immunol (2000) 30(18):2216-2225.

Useful viral antigens for example, include those derived from members of the families Adenoviradae; Arenaviridae (for example, Ippy virus and Lassa virus); Birnaviridae; Bunyaviridae; Caliciviridae; Coronaviridae; Filoviridae; Flaviviridae (for example, yellow fever virus, dengue fever virus and hepatitis C virus); Hepadnaviradae (for example, hepatitis B virus); Herpesviradae (for example, human herpes simplex virus 1); Orthomyxoviridae (for example, influenza virus A, B and C); Paramyxoviridae (for example, mumps virus, measles virus and respiratory syncytial virus); Picornaviridae (for example, poliovirus and hepatitis A virus); Poxyiridae; Reoviridae; Retroviradae (for example, BLV-HTLV retrovirus, HIV-1, HIV-2, bovine immunodeficiency virus and feline immunodeficiency virus); Rhabodoviridae (for example, rabies virus), and Togaviridae (for example, rubella virus). In one embodiment, the compositions comprise one or more antigens derived from a major viral pathogen such as the various hepatitis viruses, human immunodeficiency virus (HIV), various influenza viruses, West Nile virus, respiratory syncytial virus, influenza virus, rabies virus, human papilloma virus (HPV), Epstein Barr virus (EBV), polyoma virus, or SARS coronavirus.

Viral antigens derived from the hepatitis viruses, including hepatitis A virus (HAV), hepatitis B virus (HBV), hepatitis C virus (HCV), the delta hepatitis virus (HDV), hepatitis E virus (HEV) and hepatitis G virus (HGV), are known in the art. For example, antigens can be derived from HCV core protein, E1 protein, E2 protein, NS3 and other proteins (NS2, NS4a, NS4b, NS5a and NS5b), from HBV HbsAg antigen or HBV core antigen, and from HDV delta-antigen (see, for example, U.S. Pat. No. 5,378,814). U.S. Pat. Nos. 6,596,476; 6,592,871; 6,183,949; 6,235,284; 6,780,967; 5,981,286; 5,910,404; 6,613,530; 6,709,828; 6,667,387; 6,007,982; 6,165,730; 6,649,735 and 6,576,417, for example, describe various antigens based on HCV core protein. In one embodiment, an antigenic portion of the HCV core protein is included in the ACAS of the invention. For example, suitable antigenic portions include the first (N-terminal) 82 amino acids of the HCV core protein and the first (N-terminal) 170 amino acids of the HCV core protein.

Non-limiting examples of known antigens from the herpesvirus family include those derived from herpes simplex virus (HSV) types 1 and 2, such as HSV-1 and HSV-2 glycoproteins gB, gD and gH.

Non-limiting examples of HIV antigens include antigens derived from gp120, antigens derived from various envelope proteins such as gp160 and gp41, gag antigens such as p24gag and p55gag, as well as proteins derived from the pol, env, tat, vif rev, nef vpr, vpu and LTR regions of HIV. The sequences of gp120 from a multitude of HIV-1 and HIV-2 isolates, including members of the various genetic subtypes of HIV are known (see, for example, Myers et al., Los Alamos Database, Los Alamos National Laboratory, Los Alamos, N. Mex. (1992); and Modrow et al., J. Virol. (1987) 61:570 578).

Non-limiting examples of other viral antigens include those from varicella zoster virus (VZV), Epstein-Barr virus (EBV) and cytomegalovirus (CMV) including CMV gB and gH; and antigens from other human herpesviruses such as HHV6 and HHV7 (see, for example Chee et al. (1990) Cytomegaloviruses (J. K. McDougall, ed., Springer-Verlag, pp. 125 169; McGeoch et al. (1988) J. Gen. Virol. 69:1531 1574; U.S. Pat. No. 5,171,568; Baer et al. (1984) Nature 310:207 211; and Davison et al. (1986) J. Gen. Virol. 67:1759 1816.)

Antigens can also be derived from the influenza virus, for example, from the haemagglutinin (HA), neuramidase (NA), nucleoprotein (NP) M1 and M2 proteins. The sequences of these proteins are known in the art and are readily accessible from GenBank database maintained by the National Center for Biotechnology Information (NCBI). Suitable antigenic fragments of HA, NP and the matrix proteins include, but are not limited to, fragments comprising one or more of the haemagglutinin epitopes: HA 91-108, HA 307-319 and HA 306-324 (Rothbard, Cell, 1988, 52:515-523), HA 458-467 (J. Immunol. 1997, 159(10): 4753-61), HA 213-227, HA 241-255, HA 529-543 and HA 533-547 (Gao, W. al., J. Virol., 2006, 80:1959-1964); the nucleoprotein epitopes: NP 206-229 (Brett, 1991, J. Immunol. 147:984-991), NP335-350 and NP380-393 (Dyer and Middleton, 1993, In: Histocompatibility testing, a practical approach (Ed.: Rickwood, D. and Hames, B. D.) IRL Press, Oxford, p. 292; Gulukota and DeLisi, 1996, Genetic Analysis: Biomolecular Engineering, 13:81), NP 305-313 (DiBrino, 1993, PNAS 90:1508-12); NP 384-394 (Kvist, 1991, Nature 348:446-448); NP 89-101 (Cerundolo, 1991, Proc. R. Soc. Lon. 244:169-7); NP 91-99 (Silver et al, 1993, Nature 360: 367-369); NP 380-388 (Suhrbier, 1993, J. Immunology 79:171-173); NP 44-52 and NP 265-273 (DiBrino, 1993, ibid.); and NP 365-380 (Townsend, 1986, Cell 44:959-968); the matrix protein (M1) epitopes: M1 2-22, M1 2-12, M1 3-11, M1 3-12, M1 41-51, M1 50-59, M1 51-59, M1 134-142, M1 145-155, M1 164-172, M1 164-173 (all described by Nijman, 1993, Eur. J. Immunol. 23:1215-1219); M1 17-31, M1 55-73, M1 57-68 (Carreno, 1992, Mol Immunol 29:1131-1140); M1 27-35, M1 232-240 (DiBrino, 1993, ibid.), M1 59-68 and M1 60-68 (Eur. J. Immunol. 1994, 24(3): 777-80); and M1 128-135 (Eur. J. Immunol. 1996, 26(2): 335-39).

Other related antigenic regions and epitopes of the influenza virus proteins are also known. For example, fragments of the influenza ion channel protein (M2), including the M2e peptide (the extracellular domain of M2). The sequence of this peptide is highly conserved across different strains of influenza. An example of a M2e peptide sequence is shown in Table 2 as SEQ ID NO:21. Variants of this sequence have been identified and some examples of such variants are also shown in Table 2.

TABLE 2
M2e Peptide and Variations Thereof

| Region of M2 | Sequence | SEQ ID NO |
|---|---|---|
| 2-24 | SLLTEVETPIRNEWGCRCNDSSD | 21 |
| 2-24 | SLLTEVETPIRNEWGCRCNGSSD* | 22 |
| 2-24 | SLLTEVETPTKNEWDCRCNDSSD* | 23 |
| 2-24 | SLLTEVETPTRNGWECKCSDSSD^{‡} | 24 |
| 2-24 | SLLTEVETPTRNEWECRCSDSSD^{#} | 25 |

*see U.S. patent application No. 2006/0246092
^{‡}A/equine/Massachussetts/213/2003 (strain H3N8)
^{#}A/Vietnam/1196/04 (strain H5N1)

The entire M2e sequence or a partial M2e sequence may be used, for example, a partial sequence that is conserved across the variants, such as fragments comprising the region defined by amino acids 2 to 10, or the conserved epitope EVETPIRN [SEQ ID NO:26] (amino acids 6-13 of the M2e sequence). The 6-13 epitope has been found to be invariable in 84% of human influenza A strains available in GenBank. Variants of this sequence that were also identified include EVETLTRN [SEQ ID NO:27] (9.6%), EVETPIRS [SEQ ID NO:28] (2.3%), EVETPTRN [SEQ ID NO:29] (1.1%), EVETPTKN [SEQ ID NO:30] (1.1%) and EVDTLTRN [SEQ ID NO:31], EVETPIRK [SEQ ID NO:32] and EVETLTKN [SEQ ID NO:33] (0.6% each) (see Zou, P., et al., 2005, Int Immunopharmacology, 5:631-635; Liu et al. 2005, Microbes and Infection, 7:171-177).

Other useful antigens include live, attenuated and inactivated viruses such as inactivated polio virus (Jiang et al., J. Biol. Stand, (1986) 14:103-9), attenuated strains of Hepatitis A virus (Bradley et al., J. Med. Virol., (1984) 14:373-86), attenuated measles virus (James et al., N. Engl. J. Med., (1995) 332:1262-6), and epitopes of pertussis virus (for example, ACEL-IMUNE™ acellular DTP, Wyeth-Lederle Vaccines and Pediatrics).

Antigens can also be derived from unconventional viruses or virus-like agents such as the causative agents of kuru, Creutzfeldt-Jakob disease (CJD), scrapie, transmissible mink encephalopathy, and chronic wasting diseases, or from proteinaceous infectious particles such as prions that are associated with mad cow disease, as are known in the art.

Useful bacterial antigens include, for example, superficial bacterial antigenic components, such as lipopolysaccharides, capsular antigens (proteinaceous or polysaccharide in nature), or flagellar components and may be obtained or derived from known causative agents responsible for diseases such as Diphtheria, Pertussis, Tetanus, Tuberculosis, Bacterial or Fungal Pneumonia, Cholera, Typhoid, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borrelia, and Trichinosis.

Examples of antigens derived from gram-negative bacteria of the family Enterobacteriaceae include, but are not limited to, the S. typhi Vi (capsular polysaccharide) antigen, the E. coli K and CFA (capsular component) antigens and the E. coli fimbrial adhesion antigens (K88 and K99). Examples of antigenic proteins include the outer membrane proteins (Omps), also known as porins (Secundino et al., 2006, Immunology 117:59); related porins such as the S. typhi iron-regulated outer membrane protein (IROMP, Sood et al., 2005, Mol Cell Biochem 273:69-78), and heat shock proteins (HSPs) including, but not limited to S. typhi HSP40 (Sagi et al., 2006, Vaccine 24:7135-7141). Non-limiting examples of antigenic porins include OmpC and OmpF, which are found in numerous Salmonella and Escherichia species. Orthologues of OmpC and OmpF are also found in other Enterobacteriaceae and are suitable antigenic proteins for the purposes of the present invention. In addition, Omp1B (Shigella flexneri), OmpC2 (Yersinia pestis), OmpD (S. enterica), OmpK36 (Klebsiella pneumonie), OmpN (E. coli) and OmpS (S. enterica) may be suitable, based on conserved regions of sequences found in the porin proteins of the Enterobacteriaceae family (Diaz-Quinonez et al., 2004, Infect. and Immunity 72:3059-3062).

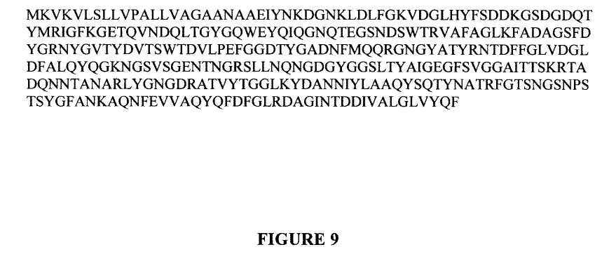

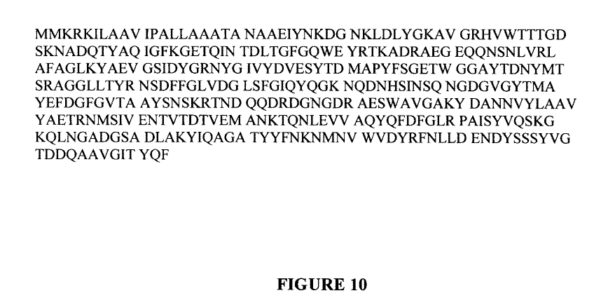

The sequences of antigenic proteins from various enterobacteria are known in the art and are readily accessible from GenBank database maintained by the National Center for Biotechnology Information (NCBI). For example, GenBank Accession No. P0A264 (also shown in FIG. 9 [SEQ ID NO:4]) and GenBank Accession No. NP_{—}804453: OmpC (S. enterica subsp. enterica serovar Typhi Ty2); GenBank Accession No. CAD05399 (also shown in FIG. 10 [SEQ ID NO:5]): OmpF precursor protein (S. enterica subsp. enterica serovar Typhi CT18); GenBank Accession No. 16761195: OmpC (S. enterica serovar Typhimurium); GenBank Accession No. 47797: OmpC (S. enterica serovar Typhi); GenBank Accession No. 8953564: OmpC (S. enterica serovar Minnesota); GenBank Accession No. 19743624: OmpC (S. enterica serovar Dublin); GenBank Accession No. 19743622: OmpC (S. enterica serovar Gallinarum); GenBank Accession No. 26248604: OmpC (E. coli); GenBank Accession No. 24113600: Omp1B (Shigella flexneri); GenBank Accession No. 16764875: OmpC2 (Yersinia pestis); GenBank Accession No. 16764916: OmpD (S. enterica Serovar Typhimurium); GenBank Accession No. 151149831: OmpK36 (Klebsiella pneumonie); GenBank Accession No. 3273514: OmpN (E. coli), and GenBank Accession No. 16760442: OmpS (S. enterica serovar Typhi).

Various tumour-associated antigens are known in the art. Representative examples include, but are not limited to, Her2 (breast cancer); GD2 (neuroblastoma); EGF-R (malignant glioblastoma); CEA (medullary thyroid cancer); CD52 (leukemia); human melanoma protein gp100; human melanoma protein melan-A/MART-1; NA17-A nt protein; p53 protein; various MAGEs (melanoma associated antigen E), including MAGE 1, MAGE 2, MAGE 3 (HLA-A1 peptide) and MAGE 4; various tyrosinases (HLA-A2 peptide); mutant ras; p97 melanoma antigen; Ras peptide and p53 peptide associated with advanced cancers; the HPV 16/18 and E6/E7 antigens associated with cervical cancers; MUC1-KLH antigen associated with breast carcinoma; CEA (carcinoembryonic antigen) associated with colorectal cancer, DKK-1 (Dickkopf-1 protein) associated with lung cancer and the PSA antigen associated with prostate cancer.

Useful allergens include, but are not limited to, allergens from pollens, animal dander, grasses, moulds, dusts, antibiotics, stinging insect venoms, as well as a variety of environmental, drug and food allergens. Common tree allergens include pollens from cottonwood, popular, ash, birch, maple, oak, elm, hickory, and pecan trees. Common plant allergens include those from rye, ragweed, English plantain, sorrel-dock and pigweed, and plant contact allergens include those from poison oak, poison ivy and nettles. Common grass allergens include Timothy, Johnson, Bermuda, fescue and bluegrass allergens. Common allergens can also be obtained from moulds or fungi such as Alternaria, Fusarium, Hormodendrum, Aspergillus, Micropolyspora, Mucor and thermophilic actinomycetes. Penicillin, sulfonamides and tetracycline are common antibiotic allergens. Epidermal allergens can be obtained from house or organic dusts (typically fungal in origin), from insects such as house mites (dermalphagoides pterosinyssis), or from animal sources such as feathers, and cat and dog dander. Common food allergens include milk and cheese (diary), egg, wheat, nut (for example, peanut), seafood (for example, shellfish), pea, bean and gluten allergens. Common drug allergens include local anesthetic and salicylate allergens, and common insect allergens include bee, hornet, wasp and ant venom, and cockroach calyx allergens.

Particularly well characterized allergens include, but are not limited to, the dust mite allergens Der pI and Der pII (see, Chua, et al., J. Exp. Med, 167:175 182, 1988; and, Chua, et al., Int. Arch. Allergy Appl. Immunol., (1990) 91:124-129), T cell epitope peptides of the Der pII allergen (see, Joost van Neerven, et al., J. Immunol., (1993) 151:2326-2335), the highly abundant Antigen E (Amb aI) ragweed pollen allergen (see, Rafnar, et al., J. Biol. Chem., (1991) 266:1229-1236), phospholipase A2 (bee venom) allergen and T cell epitopes therein (see, Dhillon, et al., J. Allergy Clin. Immunol., (1992) 42), white birch pollen (Betvl) (see, Breiteneder, et al., EMBO, (1989) 8:1935-1938), the Fel dI major domestic cat allergen (see, Rogers, et al., Mol. Immunol., (1993) 30:559-568), tree pollen (see, Elsayed et al., Scand. J. Clin. Lab. Invest. Suppl., (1991) 204:17-31) and the multi-epitopic recombinant grass allergen rKBG8.3 (Cao et al. Immunology (1997) 90:46-51). These and other suitable allergens are commercially available and/or can be readily prepared following known techniques.

Antigens relating to conditions associated with self antigens are also known to those of ordinary skill in the art. Representative examples of such antigens include, but are not limited to, lymphotoxins, lymphotoxin receptors, receptor activator of nuclear factor kB ligand (RANKL), vascular endothelial growth factor (VEGF), vascular endothelial growth factor receptor (VEGF-R), interleukin-5, interleukin-17, interleukin-13, CCL21, CXCL12, SDF-1, MCP-1, endoglin, resistin, GHRH, LHRH, TRH, MIF, eotaxin, bradykinin, BLC, tumour Necrosis Factor alpha and amyloid beta peptide, as well as fragments of each which can be used to elicit immunological responses.

Useful toxins are generally the natural products of toxic plants, animals, and microorganisms, or fragments of these compounds. Such compounds include, for example, aflatoxin, ciguautera toxin, pertussis toxin and tetrodotoxin.

Antigens useful in relation to recreational drug addiction are known in the art and include, for example, opioids and morphine derivatives such as codeine, fentanyl, heroin, morphine and opium; stimulants such as amphetamine, cocaine, MDMA (methylenedioxymethamphetamine), methamphetamine, methylphenidate, and nicotine; hallucinogens such as LSD, mescaline and psilocybin; cannabinoids such as hashish and marijuana, other addictive drugs or compounds, and derivatives, by-products, variants and complexes of such compounds.

In one embodiment of the present invention, the antigen(s) included in the ACAS are protein antigens. A protein antigen can be a full-length protein, a substantially full-length protein (for example, a protein comprising a N-terminal and/or C-terminal deletion of about 25 amino acids or less), an antigenic fragment of the protein, or a combination thereof. The full-length protein can be, when applicable, a precursor form of the protein or the mature (processed) form of the protein. The protein may be post-translationally modified, for example, a glycoprotein or lipoprotein. An antigenic fragment can comprise one, or a plurality of epitopes, and thus may range in size from a peptide of a few amino acids (for example, at least 4 amino acids) to a polypeptide several hundred amino acids in length. In one embodiment of the invention, antigenic fragments suitable for inclusion in the ACAS are at least 20 amino acids in length. In another embodiment, antigenic fragments suitable for inclusion in the ACAS are between about 20 amino acids and about 500 amino acids in length. In another embodiment, antigenic fragments suitable for inclusion in the ACAS are between about 20 amino acids and about 450 amino acids in length. In other embodiments, antigenic fragments suitable for inclusion in the ACAS are between about 20 amino acids and about 400 amino acids in length, between about 20 amino acids and about 350 amino acids in length, between about 20 amino acids and about 300 amino acids in length, between about 20 amino acids and about 250 amino acids in length, between about 20 amino acids and about 200 amino acids in length, and between about 20 amino acids and about 150 amino acids in length. In another embodiment, the protein antigen included in the ACAS is a full-length or substantially full-length protein.

### Preparation of the ACAS

aPapMV and a VLPs

PapMV is known in the art and can be obtained, for example, from the American Type Culture Collection (ATCC) as ATCC No. PV-204™. The virus can be maintained on, and purified form, host plants such as papaya (Carica papaya) and snapdragon (Antirrhinum majus) following standard protocols (see, for example, Erickson, J. W. & Bancroft, J. B., 1978, Virology 90:36-46). The selected affinity moieties can be attached to the coat protein of PapMV to form the aPapMV through reactive groups disposed on the surface of the virus, for example, via lysine, arginine, aspartate, glutamate and/or cysteine residues.

In general, the affinity moiety is chemically attached to the coat protein of the PapMV. By “chemically attached” it is meant that the affinity moieties are chemically cross-linked to the coat protein, for example, by covalent or non-covalent (such as, ionic, hydrophobic, hydrogen bonding, or the like) attachment. The affinity moiety and/or coat protein can be modified to facilitate such cross-linking as is known in the art, for example, by addition of a functional group or chemical moiety to the protein and/or antigen, for example at the C- or N-terminus or at an internal position. Exemplary modifications include the addition of functional groups such as S-acetylmercaptosuccinic anhydride (SAMSA) or S-acetyl thioacetate (SATA), or addition of one or more cysteine residues. Other cross-linking reagents are known in the art and many are commercially available (see, for example, catalogues from Pierce Chemical Co. and Sigma-Aldrich). Examples include, but are not limited to, diamines, such as 1,6-diaminohexane, 1,3-diamino propane and 1,3-diamino ethane; dialdehydes, such as glutaraldehyde; succinimide esters, such as ethylene glycol-bis(succinic acid N-hydroxysuccinimide ester), disuccinimidyl glutarate, disuccinimidyl suberate, N-(g-Maleimidobutyryloxy) sulfosuccinimide ester and ethylene glycol-bis(succinimidylsuccinate); diisocyanates, such as hexamethylenediisocyanate; bis oxiranes, such as 1,4 butanediyl diglycidyl ether; dicarboxylic acids, such as succinyldisalicylate; 3-maleimidopropionic acid N-hydroxysuccinimide ester, and the like. Many of the above-noted cross-linking agents incorporate a spacer that distances the affinity moiety from the VLP. The use of other spacers is also contemplated by the invention. Various spacers are known in the art and include, but are not limited to, 6-aminohexanoic acid; 1,3-diamino propane; 1,3-diamino ethane; and short amino acid sequences, such as polyglycine sequences, of 1 to 5 amino acids.

To facilitate covalent attachment of the one or more affinity moieties to the coat protein, a short peptide or amino acid linker can be first attached to the coat protein such that it is exposed in the surface of the PapMV and provides an appropriate site for chemical attachment of the affinity moiety. For example, short peptides comprising cysteine residues, or other amino acid residues having side chains that are capable of forming covalent bonds (for example, acidic and basic residues) or that can be readily modified to form covalent bonds as known in the art. The amino acid linker or peptide can be, for example, between one and about 20 amino acids in length. In one embodiment, the coat protein is fused with a short peptide comprising one or more lysine residues, which can be covalently coupled, for example with a cysteine residue in the moiety through the use of a suitable cross-linking agent as described above. In a specific embodiment, the coat protein is fused with a short peptide sequence of glycine and lysine residues. In another embodiment, the peptide comprises the sequence: GGKGG.

The recombinant coat proteins to be used to prepare the aVLPs of the present invention can be readily prepared by standard genetic engineering techniques by the skilled worker provided with the sequence of the wild-type protein. Methods of genetically engineering proteins are well known in the art (see, for example, Ausubel et al. (1994 & updates) Current Protocols in Molecular Biology, John Wiley & Sons, New York), as is the sequence of the wild-type PapMV coat protein (see SEQ ID NOs:1 and 2).

Isolation and cloning of the nucleic acid sequence encoding the wild-type protein can be achieved using standard techniques (see, for example, Ausubel et al., ibid.). For example, the nucleic acid sequence can be obtained directly from the PapMV by extracting RNA by standard techniques and then synthesizing cDNA from the RNA template (for example, by RT-PCR). PapMV can be purified from infected plant leaves that show mosaic symptoms by standard techniques (see, for example, Example 1 provided herein).

The nucleic acid sequence encoding the coat protein is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The coat protein can then be expressed and purified as described in more detail below.

Alternatively, the nucleic acid sequence encoding the coat protein can be further engineered to introduce one or more mutations, such as those described above, by standard in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

As noted above, when the affinity moiety is a peptide, protein or protein fragment, the coat protein can also be engineered to genetically fuse the affinity moieties to the coat protein. In order to allow presentation of the affinity moiety on the surface of the aVLP and thereby enhance immune recognition of an antigen bound to the affinity moiety, the affinity moiety is preferably fused to a region of the coat protein that is disposed on the outer surface of the aVLP. Thus the affinity moiety can be attached at, for example, the amino-(N-) or carboxy-(C-) terminus of the coat protein, or it can be attached to an internal loop of the coat protein which is disposed on the outer surface of the aVLP. In one embodiment of the present invention, the affinity moiety is genetically fused at, or proximal to, the C-terminus of the PapMV coat protein.

Methods for making fusion proteins are well known to those skilled in the art. DNA sequences encoding a fusion protein can be inserted into a suitable expression vector as noted above.

One of ordinary skill in the art will appreciate that the DNA encoding the coat protein can be altered in various ways without affecting the activity of the encoded protein. For example, variations in DNA sequence may be used to optimize for codon preference in a host cell used to express the protein, or may contain other sequence changes that facilitate expression.

One skilled in the art will understand that the expression vector may further include regulatory elements, such as transcriptional elements, required for efficient transcription of the DNA sequence encoding the coat or fusion protein. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a genetically engineered coat protein. One skilled in the art will appreciate that selection of suitable regulatory elements is dependent on the host cell chosen for expression of the genetically engineered coat protein and that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed protein. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids corresponding to expression of the nucleic acids can be removed from the expressed coat protein prior to use according to methods known in the art. Alternatively, the amino acids corresponding to expression of heterologous nucleic acid sequences can be retained on the coat protein if they do not interfere with its subsequent assembly into VLPs.

In one embodiment of the present invention, the coat protein is expressed as a histidine tagged protein. The histidine tag can be located at the carboxyl terminus or the amino terminus of the coat protein.

The expression vector can be introduced into a suitable host cell or tissue by one of a variety of methods known in the art. Such methods can be found generally described in Ausubel et al. (ibid.) and include, for example, stable or transient transfection, lipofection, electroporation, and infection with recombinant viral vectors. One skilled in the art will understand that selection of the appropriate host cell for expression of the coat protein will be dependent upon the vector chosen. Examples of host cells include, but are not limited to, bacterial, yeast, insect, plant and mammalian cells. The precise host cell used is not critical to the invention. The coat proteins can be produced in a prokaryotic host (e.g., E. coli, A. salmonicida or B. subtilis) or in a eukaryotic host (e.g., Saccharomyces or Pichia; mammalian cells, e.g., COS, NIH 3T3, CHO, BHK, 293, or HeLa cells; or insect cells). In one embodiment, the coat proteins are produced in a prokaryotic cell.

The recombinant coat proteins are capable of multimerization and assembly into VLPs. In general, assembly takes place in the host cell expressing the coat protein. The VLPs can be isolated from the host cells by standard techniques, such as those described in the Examples section provided herein. The VLPs can be further purified by standard techniques, such as chromatography, to remove contaminating host cell proteins or other compounds, such as LPS. In one embodiment of the present invention, the VLPs are purified to remove LPS. If desired, the coat proteins can be sequenced by standard peptide sequencing techniques using either the intact protein or proteolytic fragments thereof to confirm the identity of the protein.

Recombinant coat proteins and coat proteins to which affinity peptides, proteins or domains have been attached can be analysed for their ability to multimerize and self-assemble into a VLP by standard techniques. For example, by visualizing the purified protein by electron microscopy (see, for example, Example 7). VLP formation may also be determined by ultracentrifugation, and circular dichroism (CD) spectrophotometry may be used to compare the secondary structure of the recombinant or modified proteins with the WT virus (see, for example, Example 7).

Stability of the VLPs can be determined if desired by techniques known in the art, for example, by SDS-PAGE and proteinase K degradation analyses. According to one embodiment of the present invention, the PapMV VLPs of the invention are stable at elevated temperatures and can be stored easily at room temperature.

In one embodiment of the present invention, the coat proteins assemble to provide a virus or pseudovirus in the host cell and can be used to produce infective virus particles which comprise nucleic acid and fusion protein. This can enable the infection of adjacent cells by the infective virus or pseudovirus particle and expression of the fusion protein therein. In this embodiment, the host cell used to replicate the virus or pseudovirus can be a plant cell, insect cell, mammalian cell or bacterial cell that will allow the virus to replicate. In one embodiment of the present invention, the cell is a bacterial cell, such as E. coli. The cell may be a natural host cell for the virus from which the virus-like particle is derived, but this is not necessary. The host cell can be infected initially with virus or pseudovirus in particle form (i.e. in assembled rods comprising nucleic acid and a protein) or alternatively in nucleic acid form (i.e. RNA such as viral RNA; cDNA or run-off transcripts prepared from cDNA) provided that the virus nucleic acid used for initial infection can replicate and cause production of whole virus particles having the fusion protein.

When the affinity moiety is to be chemically attached to the coat protein after its assembly into a VLP, the affinity moiety may be attached by various chemical methods, as described above with respect to PapMV.

Conjugation of Antigen to the aPapMV or aVLP

The antigen can be conjugated to the aPapMV or aVLP by bringing the antigen into contact with the aPapMV or aVLP. Conjugation can occur, for example, via the formation of at least one noncovalent chemical bond, for example, a hydrogen bond, an ionic bond, a hydrophobic interaction or van der Waals interaction. Covalent attachment of the antigen to the affinity moiety is also contemplated.

Conjugation can be achieved, for example, by simple mixing of the antigen and the aPapMV or aVLP in solution with or without agitation. As noted above, an appropriate chemical agent, as is known in the art, can be added to the mixture to induce formation of covalent bounds between the aPapMV or aVLPs and the antigen, and thereby improve the strength of attachment between the aPapMV or aVLPs with the antigen. After conjugation any unconjugated antigen and/or aPapMV or aVLP and/or cross linking agent(s) can optionally be removed using standard techniques, for example, chromatography gel filtration technique that will separate the larger conjugated proteins from the unconjugated partners. Ultracentrifugation can also be used to separate the antigen from the aPapMV/aVLPs and the conjugated complex.

Optimal ratios of antigen:aPapMV/aVLP can be readily determined by the skilled worker. For example, ratios of antigen:aPapMV/aVLP of between about 10:1 and 1:10 on a weight:weight basis may be useful. In one embodiment, ratios of antigen:aPapMV/aVLP of between about 9:1 and 1:9 on a weight:weight basis are used to form the ACAS. In another embodiment, ratios of antigen:aPapMV/aVLP of between about 8:1 and 1:8 on a weight:weight basis are used to form the ACAS. In other embodiments, ratios of antigen:aPapMV/aVLP of between about 7:1 and 1:7, of about 6:1 to 1:6, and of about 5:1 and 1:5 on a weight:weight basis are used to form the ACAS.

The ability of the aPapMV or aVLP to bind its target antigen can be determined by standard techniques, for example, by flow cytometry (see, for example, Morin et al., 2007, J. Biotechnology, 128: 423-434), by electron microscopy, by a pull-down assay using ultracentrifugation or by ELISA-type assays (see examples provided herein).

### Evaluation of Efficacy

As noted above, the ACAS of the present invention are capable of inducing an immune response in an animal. The immune response may be a humoral response, a cellular response or a combination of humoral and cellular responses. The ability of the ACAS of the present invention to induce an immune response in an animal can be tested by art-known methods, such as those described below and in the Examples. For example, the ACAS can be administered to a suitable animal model, for example by subcutaneous injection or intranasally, and the development of antibodies evaluated, for example, by ELISA.

Cellular immune response can also be assessed by techniques known in the art. For example, the cellular immune response can be determined by evaluating processing and cross-presentation of an antigen conjugated to a aPapMV or aVLP to specific T lymphocytes by dendritic cells in vitro and in vivo. Other useful techniques for assessing induction of cellular immunity (T lymphocyte) include monitoring T cell expansion and IFN-γ secretion release, for example, by ELISA to monitor induction of cytokines (see, for example, Leclerc, D., et al., J. Virol, 2007, 81(3):1319-26).

In order to evaluate the efficacy of the ACASs of the present invention as vaccines, challenge studies can be conducted. Such studies involve the inoculation of groups of a test animal (such as mice) with an ACAS of the present invention by standard techniques. Control groups comprising non-inoculated animals and/or animals inoculated with a commercially available vaccine, or other positive control, are set up in parallel. After an appropriate period of time post-vaccination, the animals are challenged with the appropriate pathogen, allergen etc. Blood samples collected from the animals pre- and post-inoculation, as well as post-challenge are then analyzed for an antibody response. Suitable tests for the antibody response include, but are not limited to, Western blot analysis and Enzyme-Linked Immunosorbent Assay (ELISA). The animals can also be monitored for development of the condition associated with the antigen-containing substance or organism.

Similarly, ACASs comprising tumour-associated antigens can be tested for their prophylactic effect by inoculation of test animals and subsequent challenge by transplanting cancer cells into the animal, for example subcutaneously, and monitoring tumour development in the animal. Alternatively, the therapeutic effect of an ACAS comprising a tumour-associated antigen can be tested by administering the ACAS to the test animal after implantation of cancer cells and establishment of a tumour, and subsequently monitoring the growth and/or metastasis of the tumour.

### Immunogenic Compositions

The present invention provides for immunogenic compositions comprising one or more ACASs of the invention together with one or more non-toxic pharmaceutically acceptable carriers, diluents and/or excipients. Such compositions are suitable for use, for example, as vaccines or immunopotentiators for the prevention and/or treatment of a disease or disorder. If desired, other active ingredients, adjuvants and/or immunopotentiators may be included in the compositions. Thus, in one embodiment of the invention, the immunogenic composition may comprise one or more ACASs together with one or more PapMVs, VLPs, aPapMVs or aVLPs.

The immunogenic compositions can be formulated for administration by a variety of routes. For example, the compositions can be formulated for oral, topical, rectal or parenteral administration or for administration by inhalation or spray. The term parenteral as used herein includes subcutaneous injections, intravenous, intramuscular, intrathecal, intrasternal injection or infusion techniques. In one embodiment of the present invention, the compositions are formulated for topical, rectal or parenteral administration or for administration by inhalation or spray. In another embodiment, the compositions are formulated for parenteral administration.

The immunogenic compositions preferably comprise an effective amount of one or more ACASs of the invention. The term “effective amount” as used herein refers to an amount of the ACAS required to produce a detectable immune response in an animal. The effective amount of ACAS for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. In one embodiment of the present invention, the unit dose comprises between about 10 μg to about 10 mg of coat protein. In another embodiment, the unit dose comprises between about 10 μg to about 5 mg of coat protein. In a further embodiment, the unit dose comprises between about 40 μg to about 2 mg of coat protein. One or more doses may be used to immunize the animal, and these may be administered on the same day or over the course of several days or weeks.

As noted above, the ACAS of the present invention may comprise a plurality of antigens, and a single ACAS can thus provide a multivalent vaccine formulation. Multivalent vaccines can also be provided through the use of an ACAS comprising an antigen that is conserved amongst different members of a group of disease or disorder causing agents. Multivalent vaccine compositions that comprise a plurality of ACASs, each ACAS comprising a different antigen are also contemplated. Multivalent vaccines are useful, for example, to provide protection against more than one bacterium, virus, fungus, protozoan, parasite, cancer, an autoimmune disease, or allergen, or to provide protection against a combination of these disease or disorder causing agents. Multivalent vaccine formulations include bivalent and trivalent formulations in addition to vaccines having higher valencies. One embodiment of the present invention provides a multivalent vaccine. Another embodiment of the invention provides a multivalent vaccine that comprises an antigen that is conserved across a plurality of disease or disorder causing agents. A further embodiment provides a multivalent vaccine that comprises a plurality of (i.e. two or more) ACASs, each ACAS comprising a different antigen.

Vaccine formulations comprising a plurality of (i.e. two or more) ACASs, each ACAS comprising a different antigen, can also provide improved protection due to the higher number of epitopes in the formulation. One embodiment of the present invention thus provides for vaccine formulations comprising two or more ACASs, each ACAS comprising a different antigen. In another embodiment, there is provided a vaccine formulation comprising at least two ACAS, each ACAS including a distinct antigen derived from the same disease or disorder causing agent. In another embodiment, there is provided a vaccine formulation comprising at least two ACAS, each ACAS including a distinct portion of the disease or disorder causing agent.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the ACAS in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Compositions for oral use can also be presented as hard gelatine capsules wherein the ACAS is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Compositions formulated as aqueous suspensions contain the ACAS in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Compositions can be formulated as oily suspensions by suspending the ACAS in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may optionally be added to provide palatable oral preparations. These compositions can be preserved by the addition of an anti-oxidant such as ascorbic acid.

The immunogenic compositions can be formulated as a dispersible powder or granules, which can subsequently be used to prepare an aqueous suspension by the addition of water. Such dispersible powders or granules provide the ACAS in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Immunogenic compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Compositions can be formulated as a syrup or elixir by combining the ACAS with one or more sweetening agents, for example glycerol, propylene glycol, sorbitol or sucrose. Such formulations can also optionally contain one or more demulcents, preservatives, flavouring agents and/or colouring agents.

The immunogenic compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the composition of the present invention may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Further, one or more compounds having adjuvant activity may be optionally added to the vaccine composition. Suitable adjuvants include, for example, aluminum hydroxide, phosphate or oxide; oil-emulsions (e.g. of Bayol F® or Marcol52®); saponins, or vitamin-E solubilisate. Due to their immunostimulatory effects, PapMV or PapMV VLPs (including aPapMV and aVLPs) can also optionally be added to the immunogenic compositions as adjuvants. Opsonised vaccine compositions are also encompassed by the present invention, for example, vaccine compositions comprising antibodies isolated from animals or humans previously immunised with the vaccine. Recombinant antibodies based on antibodies isolated from animals or humans previously immunised with the vaccine could also be used to opsonise the vaccine composition.

Also encompassed by the present invention are combinations of a composition comprising an ACAS of the present invention and a commercially available vaccine.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

### Uses of the ACAS

The present invention provides for a number of applications and uses of the ACASs and the immunogenic compositions comprising same. One embodiment of the invention provides for the use of an ACAS or immunogenic composition to induce an immune response in an animal, for example, when administered as an adjuvant, immunopotentiator, immunostimulant or vaccine. The immune response may be a humoral response, a cellular response, or both. In one embodiment of the invention, the ACAS is capable of inducing a humoral response in an animal. In another embodiment, the ACAS is capable of inducing a cellular response in an animal. In a further embodiment, the ACAS is capable of inducing a cytotoxic T-lymphocyte (CTL) response in an animal. In another embodiment, the ACAS is capable of inducing both a humoral and a cellular response in an animal.

The present invention also provides for the use of the ACAS to screen for antibodies capable of binding the antigen(s) conjugated to the aPapMV or aVLP. The present invention further provides for the use of the compositions for the preparation of diagnostics and medicaments, such as adjuvants, immunopotentators, immunostimulants, vaccines and/or pharmaceutical compositions.

The ACAS of the invention is thus suitable for use in the treatment, including prevention, of a disease or disorder in an animal for which induction of an immune response is required. Dependent on the nature of the disease or disorder, treatment may require the induction of a humoral immune response, a cellular immune response or both. For example, certain infections can be effectively prevented by simply inducing a humoral response in the animal, whereas complete protection against other diseases may require induction of both humoral and cellular responses. By way of example, vaccines that induce a humoral response can be effective against typhoid fever, rabies, polio, cholera, meningitis (caused by Neisseria meningitides), hepatitis B, human metapheumovirus and some strains of influenza. Protection against other diseases or disorders is most effective when the cellular response is also induced, for example, hepatitis C, malaria, Leishmania major infection, HIV and Mycobacterium tuberculosis infection.

Accordingly, the present invention provides for the use of the ACAS as a single agent for the treatment, including prevention, of a disease or disorder, as well as for the use of the ACAS as a component of a combination vaccine or therapy for those diseases/disorders that require a more complex immune response. Such combinations may include, for example, additional vaccines, adjuvants and/or antigens. In this context, the ACAS can act as an immunopotentiator or as an adjuvant to enhance the immune response in the animal being treated. The ACAS can also be used to prime the immune system prior to the administration of a second vaccine. Administration of the ACAS in this context can, therefore, occur prior to, after or concurrent with the administration of the secondary vaccine, adjuvant or antigen.

The ACAS of the invention are suitable for use in humans as well as non-human animals, including domestic and farm animals. The administration regime for the composition need not differ from any other generally accepted vaccination programs. For example, as noted above, a single administration of the ACAS in an amount sufficient to elicit an effective immune response may be used or, alternatively, other regimes of initial administration of the ACAS followed by boosting with antigen alone or with the ACAS may be used. Similarly, boosting with either the ACAS or antigen may occur at times that take place well after the initial administration if antibody titres fall below acceptable levels. The exact mode of administration of the ACAS will depend for example on the components of the ACAS, the animal to be treated and the desired end effect of the treatment. Appropriate modes of administration can be readily determined by the skilled practitioner.

When the regime comprises the administration of an ACAS and an additional antigen or antigens, the ACAS component can be administered concomitantly with the antigen(s), or it can be administered prior or subsequent to the administration of the antigen(s), depending on the needs of the subject in which an immune response is desired.

One embodiment of the present invention provides for the use of an ACAS of the invention in conjunction with a conventional vaccine. In accordance with this embodiment, the ACAS may be administered concomitantly with the conventional vaccine (for example, by combining the two compositions), or it can be administered prior or subsequent to the administration of the conventional vaccine.

The ACAS of the invention can be used prophylactically, for example to prevent infection by a virus, bacteria or other infectious particle, or development of a tumour, or it may be used therapeutically to ameliorate the effects of a disease or disorder associated with an infection, autoimmune or allergic reaction, drug addition or a cancer. In one embodiment of the invention, the ACAS is used prophylactically to prevent a disease or disorder in an animal. The animal to which the ACAS is administered may be a human, or non-human animal, such as, a cow, pig, horse, goat, sheep, dog, cat, chicken, duck, turkey, non-human primate, guinea pig, rabbit, ferret, rat, hamster, mouse, fish or bird.

As noted above, the ACAS of the invention can be used in the prevention or treatment of a variety of diseases or disorders depending on, for example, pathway requiring activation to treat the ailment, and the antigen selected for inclusion in, or use with, the composition. Non-limiting examples include influenza (using antigens from various influenza viruses), typhoid fever (using antigens from S. typhi), HCV infections (using HCV antigens), HBV infections (using HBV antigens), HAV infections (using HAV antigens), delta hepatitis virus (using HDV antigens), hepatitis E virus (using HEV antigens), hepatitis G virus (using HGV antigens), herpes simplex virus (using HSV antigens), varicella zoster virus (using VZV antigens), Epstein-Barr virus (using EBV antigens), cytomegalovirus (using CMV antigens), other human herpesviruses (for example, using HHV6 or HHV7 antigens), HIV infections (using HIV antigens), polio (using poliovirus antigens), diphtheria (using antigens derived from diphtheria toxin), allergic reactions (using various allergens) and cancer (using various tumour-associated antigens). Other uses include, for example, prevention or treatment of inflammatory diseases (for example, arthritis) and infections by avian flu virus, human respiratory syncytial virus, Dengue virus, measles virus, human papillomavirus, pseudorabies virus, swine rotavirus, swine parvovirus, Newcastle disease virus, foot and mouth disease virus, hog cholera virus, African swine fever virus, infectious bovine rhinotracheitis virus, infectious laryngotracheitis virus, La Crosse virus, neonatal calf diarrhea virus, bovine respiratory syncytial virus, bovine viral diarrhea virus, Mycoplasma hyopneumoniae, Streptococcal bacteria, Gonococcal bacteria, Enterobacteria and parasites (for example, leishmania or malaria).

The invention also provides for the use of the ACAS to generate antibodies that prevent and/or attenuate diseases or disorders caused or exacerbated by “self” gene products. Examples of such diseases or conditions include graft versus host disease, IgE-mediated allergic reactions, anaphylaxis, adult respiratory distress syndrome, Crohn's disease, allergic asthma, acute lymphoblastic leukemia (ALL), diabetes, non-Hodgkin's lymphoma (NHL), Graves' disease, systemic lupus erythematosus (SLE), inflammatory autoimmune diseases, myasthenia gravis, immunoproliferative disease lymphadenopathy (IPL), angioimmunoproliferative lymphadenopathy (AIL), immunoblastive lymphadenopathy (IBL), rheumatoid arthritis, diabetes, multiple sclerosis, Alzheimer disease, osteoporosis, and autoimmune conditions associated with certain infections including rheumatic fever, scarlet fever, lyme disease, and infectious polyarthritis.

The invention further provides for the use of the ACAS for stimulating immune responses against compounds such as hormones, drugs and toxic compounds. Immune responses against a variety of drugs, hormones and toxic compounds are used to protect an individual at risk of exposure to such compounds, as therapy in an individual exposed to such compounds, or to prevent or treat addictions to such compounds.

The present invention also provides for the use of the ACAS as a screening agent, for example, to screen for antibodies to the antigens conjugated to the ACAS. The ACAS can be readily adapted to conventional immunological techniques such as an enzyme-linked immunosorbant assay (ELISA) or Western blotting and is thus useful in diagnostic and research contexts.

### Kits

The present invention additionally provides for pharmaceutical kits comprising one or more ACASs. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the ACAS.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

Screening kits containing one or more ACASs of the invention for use in antibody detection are also provided. The kits can be diagnostic kits or kits intended for research purposes. Individual components of the kit would be packaged in separate containers and, associated with such containers, can be a notice in the form prescribed by a governmental agency regulating the manufacture, use or sale of biological products, which notice reflects approval by the agency of manufacture, use or sale of the biological product. The kit may optionally contain instructions or directions outlining the method of use or administration regimen for the vaccine.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Adjuvant Effect of PapMV PapMV Purification

PapMV was purified by differential centrifugation from infected papaya leaves that showed mosaic symptoms. Infected leaves (100 g) were ground in 100 mL 50 mM Tris-HCl (pH 8.0) containing 10 mM EDTA in a commercial blender. The ground leaves were filtered through cheesecloth, 1% of Triton X-100 was added to the filtrate, and the filtrate was stirred gently for 10 min. Chloroform was added drop by drop to a volume equivalent to one-quarter of the volume of the filtrate. The solution was stirred for an additional 30 min at 4° C. and centrifuged for 20 min at 10 000 g to remove the precipitate. The supernatant was subjected to high-speed (100 000 g) centrifugation for 120 min. The viral pellet was suspended and subjected to another high-speed centrifugation through a sucrose cushion (30% sucrose) at 100 000 g for 3.5 h. The final viral pellet was suspended in 10 mL of 50 mM Tris (pH 8.0). If color persisted, an additional clarification with chloroform was performed. The purified virus was collected by ultracentrifugation.

### Antigens

LPS-free OVA Grade VI was purchased from Sigma-Aldrich Chemical Co, St Louis, Mo. Hen egg white lysozyme (HEL) was purchased from Research Organics Inc. Cleveland, Ohio. LPS from E. coli O111:B4 was purchased from Sigma-Aldrich, St Louis, Mo.

### Immunizations

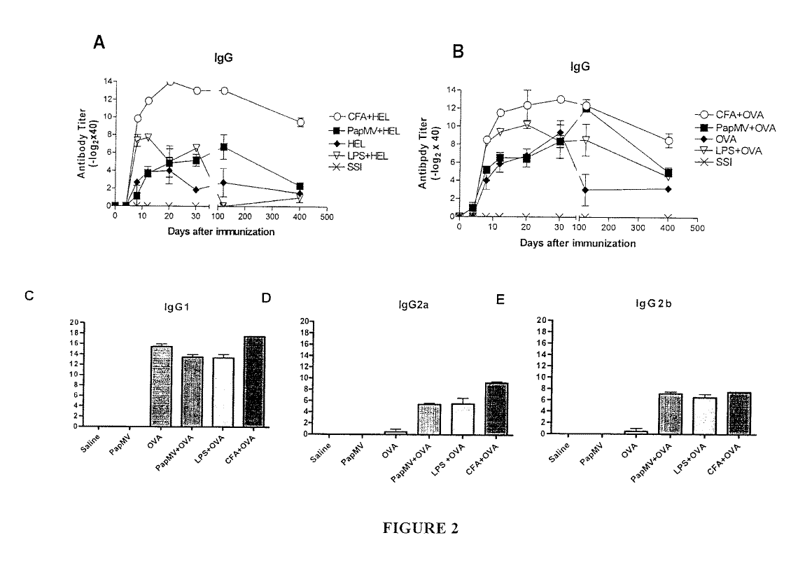

BALB/c mice, 6-8 weeks old, were bred and kept under the animal facilities of the Experimental Medicine Department, Faculty of Medicine, National Autonomous University of Mexico (UNAM), and were cared for in conformity with good laboratory practice guidelines. To study the effects of adjuvant, groups of mice were immunized i.p. on day 0 with 2 mg of OVA or HEL alone or with 30 mg of PapMV, CFA 1:1 (v/v), or 5 mg of LPS from E. coli O111:B4 (Sigma-Aldrich). Control mice were injected with saline solution only. Blood samples were collected from the retro-orbital sinus at various times, as indicated in FIG. 2. Individual serum samples were stored at −20° C. until analysis. Three mice were used in each experiment.

### Determination of Antibody Titers by ELISA

High-binding 96-well polystyrene plates (Corning®, New York, N.Y.) were coated with 1 mg/mL of PapMV, 100 mg/mL of HEL, or 150 mg/mL OVA in 0.1 M carbonate-bicarbonate buffer (pH 9.5). Plates were incubated for 1 h at 37° C. and then overnight at 4° C. Before use the next morning, plates were washed three times in PBS (pH 7.2) containing 0.05% Tween-20 (PBS-T) (Sigma-Aldrich). Nonspecific binding was blocked with 5% nonfat dry milk diluted in PBS (PBS-M) for 1 h at 37° C. After washing, mice serum was diluted 1:40 in PBS-M, and 2-fold serial dilutions were added to the wells. Plates were incubated for 1 h at 37° C. and then washed four times with PBS-T. Peroxidase-conjugated rabbit anti-mouse IgM (optimal dilution 1:1000) IgG, IgG1, IgG2a, IgG2b antibodies (Zymed, San Francisco, Calif.) or IgG3 (optimal dilution 1:3000)(Rockland, Gilbertsville, Pa.) was added, and the plates were incubated for 1 h at 37° C. and washed three times with PBS-T. Orthophenylenediamine (0.5 mg/mL; Sigma-Aldrich) in 0.1 M citrate buffer (pH 5.6) containing 30% hydrogen peroxide was used as the enzyme substrate. The reaction was stopped with 2.5 NH₂SO₄, and the absorbance was determined at 490 nm using an automatic ELISA plate reader (Dynex Technologies MRII, Chantilly, Va., USA) with BIOLINX 2.22 software. Antibody titers are given as -log 2 dilution×40. A positive titer was defined as 3 SD above the mean value of the negative control.

### Results

The translation of innate immune response into antibody response is observed when adjuvants are co-administered to poor immunogenic vaccines. Adjuvants are substances capable of strengthen or augment the antibody or cellular immune response against an antigen. To determine whether PapMV is an adjuvant that can promote a long-lasting antibody response to other antigens, BALB/c mice were immunized with OVA or HEL either alone or together with the following adjuvants: PapMV, CFA, or LPS. The IgG antibody titer specific for OVA or HEL was measured by ELISA at the time points indicated (FIGS. 2A and B). PapMV adjuvant effect was observed on the total IgG response to OVA and HEL in immunized animals. An adjuvant effect induced by PapMV was observed for HEL on day 30 after immunization, when the antibody titer increased 8-fold compared with the antibody titer induced by HEL alone. This difference in antibody titer was maintained until day 120 but not on day 400 (FIG. 2A). Although LPS induced an adjuvant effect only in the first 30 days after immunization, CFA showed the strongest adjuvant effect from day 8 to the end of the experiment on day 400 after immunization (FIG. 2A). For immunization with OVA, the adjuvant effect on total IgG antibody titers was observed only until day 120 after the first immunization, after which antibody titer decreased with time and was 4-fold higher compared with OVA on day 400 (FIG. 2B). Further analysis was performed on day 20 to identify which IgG subclasses were induced by OVA and OVA coimmunized with adjuvants (where PapMV did not show an adjuvant effect on the total IgG response). PapMV, LPS, and CFA induced OVA-specific IgG2a and IgG2b antibody titers, whereas OVA alone induced only IgG1-specific antibody titers (FIG. 2C-E). No adjuvant effect for IgG1 was observed when OVA was coimmunized with any of the adjuvants used. These results show that PapMV, LPS, and CFA induce an adjuvant effect on the IgG subclass responses to OVA. Moreover, PapMV exhibits adjuvant properties that induce a long-lasting increase in specific antibody titers to model antigens. Taken together, these data suggest that PapMV has intrinsic adjuvant properties that may have mediated the translation of the innate response into the antigen-specific long-lasting antibody response observed.

### Example 2

### Purification of Salmonella typhi Porin Proteins

The following purification procedure was used for purification of OmpC and OmpF. The purification procedure is based on that described by Secundino et al. (2006), Immunology 117:59.

The two proteins were co-purified from Salmonella typhi. Individual purification of OmpC and OmpF was achieved using knock-out mutants of S. typhi in which either OmpC [STYC171 (OmpC⁻)] or OmpF [STYF302 (OmpF⁻)] open reading frames are interrupted. The procedure for purification of the individual proteins from the knock-out mutated forms of the bacteria was followed as for the co-purification. This procedure is outlined below.

The bacterial strain, Salmonella typhi 9,12,Vi:d (ATCC 9993) was grown in Minimal medium A supplemented with yeast extract, magnesium and glucose at 37° C., 200 rpm. The formula for 10 L Minimal medium A supplemented with yeast extract, magnesium and glucose is: 5.0 g of dehydrated Na-Citrate (NaC₆H₅O₇:2H₂O), 31.0 g NaPO₄ monobasic (NaH₂PO₄), 70.0 g NaPO₄ dibasic (Na₂HPO₄), 10.0 g (NH₄)₂SO₄, 200 mL yeast extract solution 5% (15.0 g in 300 mL). 1.434 L medium was distributed per 4 L Erlenmeyer flask. Sterilization was performed at 121° C., 15 lbs pression/in², 15 min. to each flask was then added: 6.0 mL of sterile MgSO₄ solution 25% and 60.0 mL of glucose solution 12.5%. The flask was inoculated with an overnight culture of S. typhi and when the OD₅₄₀ reached 1.0, incubation was stopped and the culture centrifuged at 7,500 rpm for 15 min at 4° C. The pellet was resuspended in 100 mL final of Tris-HCl pH 7.7 (6.0 g Tris-base/L) and the biomass was sonicated for 90 min on ice and then centrifuged at 7,500 rpm for 20 min at 4° C. To each 10 mL of supernatant was added: 2.77 mL MgCl₂ 1M, 25 ml RNaseA (10,000 U/mL), 25 ml DNaseA (10,000 U/mL). The mixture was then incubated at 37° C. and 120 rpm for 30 min.

Porin extraction from the mixture was performed by first ultra centrifuging the mixture at 45,000 rpm, 45 min, 4° C. and retaining the pellet. The pellet was then resuspended in 10 mL Tris-HCl-SDS 2% followed by homogenization. An incubation step was next performed at 32° C., 120 rpm, 30 min. and the mixture was ultracentrifuged fat 40,000 rpm, 30 min, 20° C. and the pellet retained. The pellet was resuspended in 5 mL Tris-HCl-SDS 2% followed by homogenization and an incubation step at 32° C., 120 rpm, 30 min. Another ultracentrifugation step at 40,000 rpm, 30 min, 20° C. was performed and the pellet retained. The pellet was resuspended in 20 mL Nikaido buffer-SDS 1% followed by homogenisation. [For IL of Nikaido buffer: 6.0 g Tris-base, 10.0 g SDS, 23.4 g NaCl, 1.9 g EDTA was dissolved in water and the pH adjusted to pH 7.7. 0.5 mL β-mercaptoethanol solution was then added]. The mixture was then incubated at 37° C., 120 rpm, 120 min. Finally, the mixture was ultracentrifuged at 40,000 rpm, 45 min, 20° C. and the supernatant, which contained the porin extract, was recovered.

The porins were purified from the supernatant using fast protein liquid chromatography (FPLC). 0.5× Nikaido buffer (see above) without β-mercaptoethanol was employed during the purification process. The proteins were separated using a Sephacryl S-200 (FPLC WATERS 650 E) with a Flux speed: 10 mL/min. The column was loaded with 22 mL of supernatant. Eluted fractions were monitored at 260 and 280 nm. The main peak, which contained the purified porins, was retained and stored at 4° C. The purified porins were stable for long period (over one year).

### Results

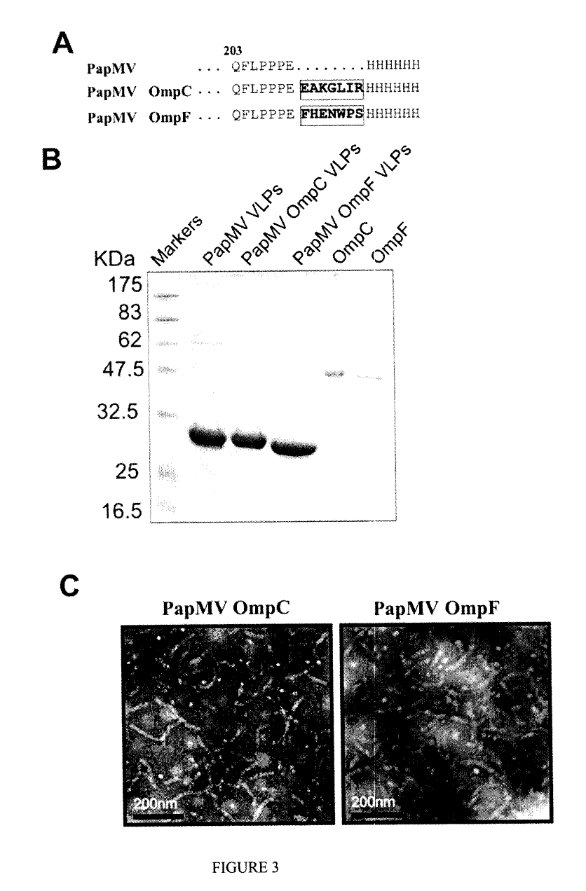

FIG. 3B shows the SDS-PAGE profile of the porins, OmpC and OmpF, purified by the procedure described above.

### Example 3

### Production and Engineering of PapMV VLPs Comprising Affinity Peptides for OmpC or OmpF

### Selection of Affinity Peptides

Specific peptides against purified OmpC and OmpF were selected using the Ph.D-7 Phage Display Peptide Library Kit (New England Biolabs, Inc.). The protocol followed was an in vitro selection process known as “panning,” which was conducted according to the manufacturer's protocol. Briefly, 2×10¹¹ phage were added to 10 μg of purified OmpC or OmpF bound to the base of the wells of an ELISA plate and the contents of the well gently mixed at room temperature for 1 hour. Unbound phage were eluted with 1 ml of 200 mM Glycine-HCl (pH 2.2), by incubating for 10 min at room temperature. To neutralize the supernatant, and to avoid killing the phage, 150 μl of 1M Tris-HCl (pH 9.1) was added. The eluted phage were then amplified and taken through additional binding/amplification cycles to enrich the pool in favour of binding sequences. The wash buffer contained 0.1% of Tween 20 for the first round of panning and was increased to 0.5% for subsequent rounds. Selected phage were amplified in E. coli ER2738 between each panning round. The cycle was repeated 3 times to select those peptides with the highest affinity for the respective porin proteins. The peptides thus identified are shown in Table 3.

TABLE 3
Sequence and Frequency of Occurrence of OmpC
and OmpF Affinity Peptides

| Target Protein | Sequence of Peptide | Frequency | SEQ ID NO |
|---|---|---|---|
| OmpC | SLSLIQT | ⅛ | 9 |
| OmpC | EAKGLIR | 6/8 | 10 |
| OmpC | TATYLLD | ⅛ | 11 |
| OmpF | FHENWPS | ⅗ | 12 |
| OmpF | FHEFWPT | ⅖ | 13 |

### Engineering, Expression and Purification of the High Avidity PapMV VLPs Fused to the Selected Affinity Peptides

One affinity peptide was selected from those identified in the above panning process for each porin, OmpC and OmpF. The corresponding DNA sequence was cloned at the C-terminus of PapMV coat protein (CP). PapMV CP CPΔN5 (Tremblay, M-H., et al., 2006, FEBS J., 273:14-25) was used as the template. The sequence encoding each selected peptide was introduced using PCR and cloned into the pET-3D expression vector (Stratagene, La Jolla, Calif.). In brief, the forward oligonucleotide (SEQ ID NO:34; Table 4) and the oligonucleotide PapOmpC (SEQ ID NO:35; Table 4) were used in the PCR reaction with the PapMV CP gene PapMV CP CPΔN5 as template.

The resulting PCR fragment harbours a fusion of the peptide EAKGLIR at the C-terminus of the PapMV CP. Using the same approach, the forward oligonucleotide (SEQ ID NO:34; Table 4) and the oligonucleotide PapOmpF (SEQ ID NO:36; Table 4) were used to introduce a fusion of the peptide FHENWPS at the C-terminus of the PapMV CP by PCR. The two respective PCR fragments were digested with the restriction enzymes NcoI and BamHI and cloned into the pET 3-D vector digested with the same enzymes. Clones were sequenced to verify that the peptides were in frame with the PapMV CP.

TABLE 4
Primers used to introduce the OmpC or OmpF
affinity peptide at the C-terminus of PapMV
CP by PCR.

| | | SEQ |
|---|---|---|
| Primer | | ID |
| Name | Sequence | NO: |
| PapN- | 5′ATCGCCATGGCATCCACACCCAACATAGCCTTCCC | 34 |
| terminus | CGCCATCACC 3′ | |
| (Forward) | | |
| PapOmpC | 3′GGTTAAGGAAGGTGGGGGGCTTCTCCGCTTCCCCA | 35 |
| (Reverse) | ACTAAGCATGGTAGTGGTAGTGGTAATCATTCCTAGG | |
| | TGAC 5′ | |
| PapOmpF | 3′GGTTAAGGAAGGTGGGGGGCTTAAAGTACTCTTAA | 36 |
| (Reverse) | CCGGAAGCGTGGTAGTGGTAGTGGTAATCATTCCTAG | |
| | GTGAC 5′ | |

Engineered PapMV CPs comprising the affinity peptide were expressed in E. coli BL21 RIL as described previously (Tremblay, M-H., et al., 2006, FEBS J, 273:14-25; Secundino et al., 2006, Immunology 117:59). Briefly, the bacteria were lysed through a French Press and loaded onto a Ni²⁺ column, washed with 10 mM Tris-HCl 50 mM Imidazole 0.5% Triton X100 pH8, then with 10 mM Tris-HCl, 50 mM Imidazole, 1% Zwittergent pH8 to remove endotoxin contamination. The eluted proteins were subjected to high-speed centrifugation (100 000 g) for 120 min in a Beckman 50.2 TI rotor. The VLP pellet was resuspended in endotoxin-free PBS (Sigma). Proteins were filtered using 0.45 μM filters before use. The purity of the proteins was determined by SDS-PAGE. The amount of protein was evaluated using the BCA protein kit (Pierce). The level of LPS in the purified protein was evaluated with the Limulus test according to the manufacturer's instructions (Cambrex) and was below 0.005 endotoxin units (EU)/μg of protein.

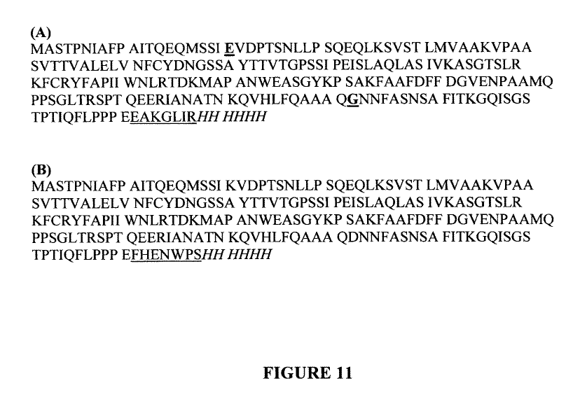

The sequences of the two PapMV coat proteins are shown in FIG. 11 (SEQ ID NO:6—PapMV coat protein including the OmpC affinity peptide, and SEQ ID NO:7—PapMV coat protein including the OmpF affinity peptide). Two amino acid differences were observed in the coat protein sequence of PapMV OmpC as compared to the wild-type (in bold and underlined in FIG. 11), which were likely introduced during the PCR reaction.

### ELISA

For each experiment, 10 μg of the respective target protein (OmpC or OmpF) was used to coat an ELISA plate. Increasing amounts of the respective PapMV VLPS were used for the binding assay. The affinity of the VLPs for their target was revealed using polyclonal mouse antibodies directed to the PapMV CP and a secondary anti-mouse antibody coupled to peroxidase.

### Results

### Selection of Affinity Peptides

Phage display was used to select specific peptides binding to OmpC or OmpF. Eight phage that bound to OmpC and five phage that bound to OmpF were sequenced. The sequences and frequency of occurrence of these peptides is shown in Table 3. The peptide EAKGLIR [SEQ ID NO:10] showed the highest frequency and, therefore, was selected as the affinity peptide to OmpC. The peptide FHENWPS [SEQ ID NO:12] was the most frequent in the OmpF screening and was, therefore, selected as the affinity peptide to OmpF. Interestingly, both affinity peptides to OmpF were homologous since 5 out of 7 amino acids were identical and found in the same position in the affinity peptides.

### Synthesis of High Avidity PapMV VLPs

The peptide sequences EAKGLIR [SEQ ID NO:10] and FHENWPS [SEQ ID NO:12] were fused at the C-terminus of the PapMV coat protein (FIG. 3A). The fusion peptide was followed by a 6×H tag to facilitate the purification process (Tremblay, M-H., et al., 2006, FEBS J., 273:14-25). The recombinant constructs were expressed in E. coli and purified by affinity chromatography on a Ni²⁺ column. The proteins were eluted using 500 mM imidazole, dialysed and ultracentrifuged at 100,000 g to pellet the VLPs (FIG. 3B). Electron microscopy (EM) observations confirmed that the addition of the respective peptides at the C-terminus of the PapMV CP did not affect the ability of the protein to self-assemble into VLPs (FIG. 3C). The lengths of the VLPs are variable, with a size range of 201±80 nm. A 201 nm length protein represents 560 copies of the CP presenting the peptide in a repetitive fashion.

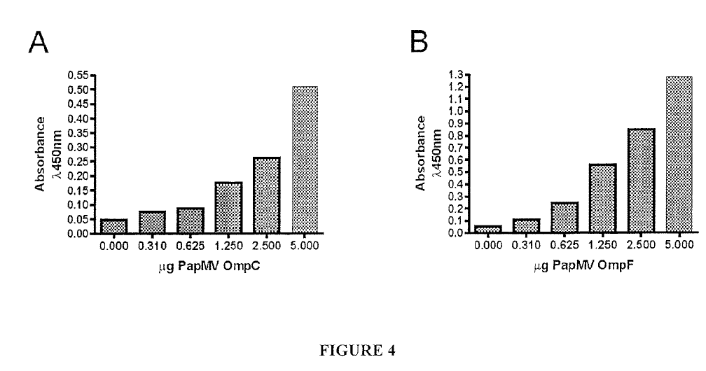

The high avidity of each of the PapMV VLPs to their respective antigen was shown by an ELISA-type binding assay. For both VLPs (“PapMV OmpC” and “PapMV OmpF”), binding to their respective antigen was clearly demonstrated and increased with the amount of VLPs used in the assay (FIGS. 4A and B). It was, therefore, assumed that PapMV VLPs will bind to the cognate antigen to form a complex when mixed in a 1:1 ratio (weight by weight) in solution.

### Example 5

### Immunization Against Salmonella typhi with Affinity-Conjugated PapMV VLP-OmpC and PapMV VLP-OmpF

### Mice

Female BALB/c mice 6-8 weeks old (Harlan, Mexico or Charles River, Canada) were used and kept in the animal facilities of the Experimental Medicine Department, Medicine Faculty, National Autonomous University of Mexico (UNAM).

### Challenge Assay

Mice (10 per group) were immunized intraperitoneally (i.p) (day 0) in the absence of external adjuvant with 10 μg OmpC, 10 μg OmpC+10 μg PapMV OmpC, 10 μg OmpF, 10 μg OmpF+10 μg PapMV OmpF, 10 μg PapMV OmpC, 10 μg PapMV OmpF or saline (SSI). On day 15, mice received a boost i.p with 10 μg OmpC or 10 μg OmpF, respectively, without adjuvant. PapMV OmpC and OmpC were mixed together between 1 and 24 hours prior to immunization and stored at 4° C.

On day 25 or 140 the mice were challenged i.p with 100 or 500 LD₅₀ of Salmonella typhi (ATCC 9993) resuspended in 500 μL TE buffer (50 mM Tris, pH 7.2, 5 mM EDTA) containing 5% gastric mucin (Sigma). Protection was defined as the percentage survival 10 days following the challenge. 1 LD₅₀ was determined at 90 000 CFU.

### Immunizations

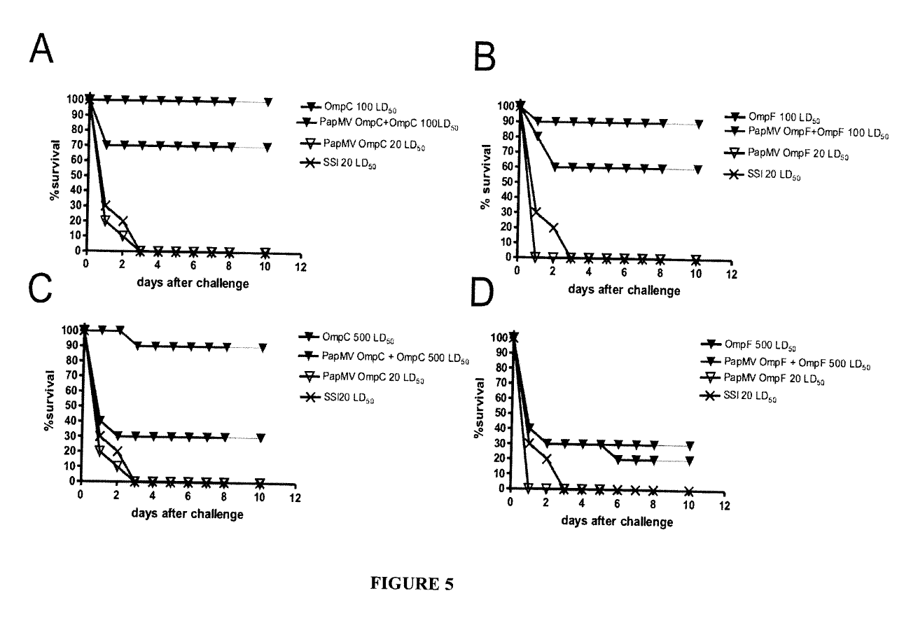

Groups of 5 mice were immunized (day 0) intraperitoneally (i.p) in the absence of external adjuvant with 10 μg OmpC, 10 μg OmpC+10 μg PapMV OmpC, 10 μg PapMV OmpC or isotonic saline solution (ISS). On day 15, mice received a boost i.p with 10 μg OmpC without adjuvant. Blood samples were collected from the jugular vein at various times as indicated in FIGS. 5 to 7. Individual serum samples were stored at −20° C. until analysis.

### ELISA

High binding 96-well polystyrene plates (Nunc) were coated with 10 μg/mL of OmpC in 0.1M carbonate-bicarbonate buffer ph 9.5. Plates were incubated for 1 hour at 37° C. followed by overnight at 4° C. Plates were washed four times with distilled H₂O-0.1% Tween 20. Non-specific binding was blocked with blocking buffer (PBS pH 7.4-2% BSA (Sigma)) for 1 hour at 37° C. After washing, pooled mice sera were diluted 1:40 in blocking buffer and two-fold serial dilutions were added to the wells. Plates were incubated 1.5 hours at 37° C., followed by four washes. HRP-conjugated goat anti-mouse IgG₁, IgG₂ₐ, IgG2b (Jackson Immunochemicals) or IgG3 (Rockland) (1:10 000) was added and incubated 1 hour at 37° C. followed by four washes. As the detection system, TMB peroxidase substrate (Fitzgerald) was used. After incubation in the dark for 10 minutes at 37° C. the reaction was stopped with 2.5N H₂SO₄ and the absorbance was determined at 450 nm using an automatic ELISA plate reader. Antibody titers are given as -log₂ dilution ×40. Positive titers were defined as 3 SD above the mean values of the negative controls.

### Passive Immunization and Challenge

Groups of 5 mice were immunized i.p (day 0) in the absence of external adjuvant with 10 μg OmpC, 10 μg OmpC+10 μg PapMV OmpC, 10 μg PapMV OmpC or isotonic saline solution (SSI). On day 15, mice received a boost i.p with 10 μg OmpC without adjuvant. Cardiac puncture was performed on day 23 and serum samples from each group were pooled and stored at −20° C. Naïve mice (5 per group) received i.p 200 μL of the pooled complement-inactivated immune sera. Three hours after transference mice were challenged with 100 LD₅₀ of Salmonella typhi resuspended in mucin, as described above. Protection was defined as the percentage survival 10 days following the challenge.

### Results

PapMV VLPs Improve the Protection Capacity of porins

The purified proteins OmpC and OmpF were previously shown to provide protection against S. typhi challenge in mice, with OmpC alone providing 60% protection against 100 LD₅₀ (Secundino et al., 2006, Immunology 117:59). To improve the immunogenicity of each of the porins, OmpC and OmpF, respectively, vaccine formulations comprising PapMV VLPs were prepared. Two different preparations, PapMV OmpC VLPs+OmpC and PapMV OmpF VLPs+OmpF, were tested in mice for their capacity to protect mice toward 100 and 500 LD₅₀ of S. typhi and the results compared with those obtained with mice immunised with OmpC or OmpF alone. The ratio between the PapMV VLPs and their respective porin was maintained at 1:1.

Addition of PapMV OmpC VLPs to OmpC improved the protection capacity of OmpC from 70% to 100% with a challenge of 100 LD₅₀ of S. typhi (FIG. 5A). This improvement of the protection efficacy was even greater when mice were challenged with 500 LD₅₀, with the protection observed increasing from 30% to 90% when OmpC was combined with PapMV OmpC VLPs (FIG. 5C). Similarly, PapMV OmpF VLPs improved the protection capacity of OmpF from 60% to 90% with a challenge of 100 LD₅₀ of S. typhi (FIG. 5B), however, only a minor difference was observed when the challenge was conducted with 500 LD₅₀ of S. typhi (FIG. 5D). The results suggest that OmpC is a better antigen than OmpF for protection against S. typhi challenge. In both cases, PapMV VLPs considerably improved the protective capacity of the porins.

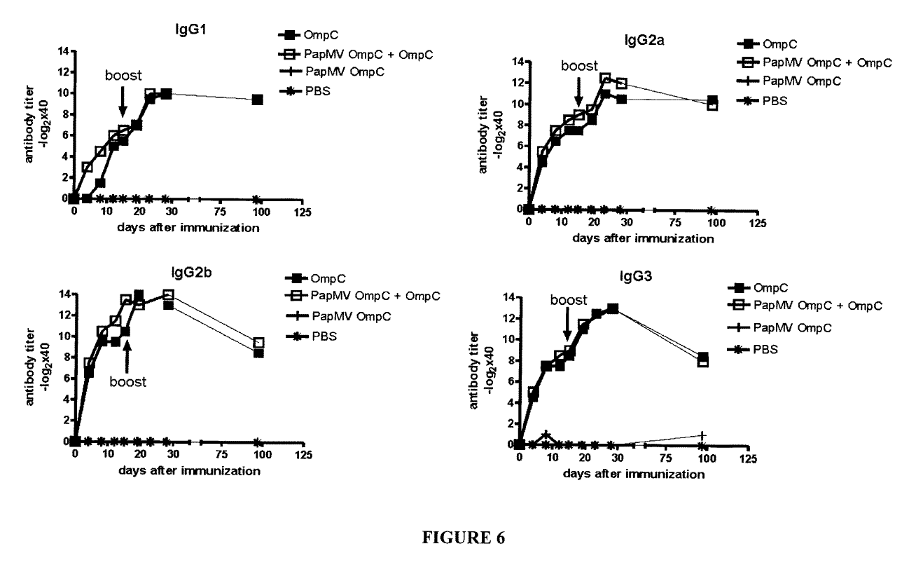

To determine if PapMV VLPs improved antibody titers to OmpC, the IgG titers of mice vaccinated with 10 μg OmpC or with the conjugated vaccine containing 10 μg OmpC and 10 μg PapMV OmpC VLPs were measured. No significant difference was found in the titers of the different IgG isotypes IgG1, IgG2a, IgG2b and IgG3 with either treatment (FIG. 6) suggesting that the improvement of the protection observed with PapMV VLPs may be related to an improvement in the CTL response and/or in the binding efficacy of the antibodies in neutralizing S. typhi infection, rather than an increase of production of antibodies per se.

Vaccination with the PapMV VLPs Improves the Memory Response to the Porins

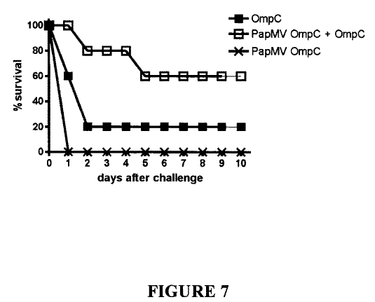

To evaluate the memory response of the vaccine preparation comprising the PapMV VLPs in combination with OmpC, mice were immunised twice at two-week intervals with either OmpC alone, or with the vaccine preparation comprising PapMV OmpC VLPs and OmpC, followed with a boost at day 15 with OmpC alone. At day 140, the mice were challenged with 100 LD₅₀ of S. typhi. The results clearly show that priming with the vaccine preparation comprising PapMV OmpC VLPs and OmpC significantly improved (3 times improvement) the protection capacity of vaccinated mice (FIG. 7). This experiment thus demonstrates that PapMV VLPs not only improve the protection of mice to S. typhi challenge, but also provide a better memory response.

### Example 5

### Protective Capacity of a Combination of PapMV and OmpC Against S. typhi

### PapMV Purification

PapMV was purified as described in Example 1.

### Protection Assay

BALB/c mice (groups of 10) were immunized i.p. on day 0 with 10 μg of OmpC or 10 μg of OmpC that had been incubated previously for 1 h at 4° C. with 30 μg of PapMV. A boost on day 15 was performed with 10 μg of OmpC alone. Control mice were injected with saline only. On day 21, the mice were challenged with 100 and 500 LD50 of S. typhi (STYC302 DompF strain) suspended in 5% mucin (as described above) and the survival rate was monitored for 10 days after the challenge, as described previously (Isibasi et al., 1992, Vaccine 10:811-813; Isibasi et al., 1988, Infect. Immun. 56:2953-2959).

### Results

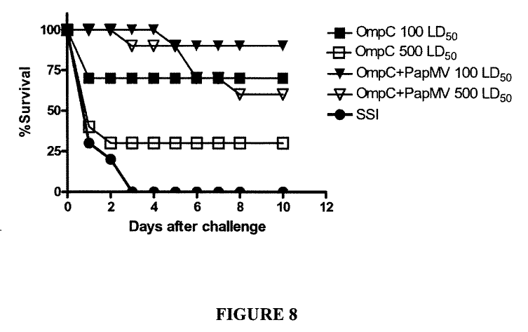

To test the adjuvant capacity of PapMV virus isolated from infected plants in increasing the protection provided by OmpC, mice immunized with OmpC and mice immunized with OmpC mixed with PapMV purified virus and subsequently challenged with S. typhi were compared. A survival rate 20% to 30% higher after challenge with either 100 LD₅₀ or 500 LD₅₀ of S. typhi was observed when OmpC mixed with PapMV purified virus was employed as compared to OmpC alone (FIG. 8). Co-administration of PapMV and S. typhi OmpC porin can thus be seen to increase the protective capacity against S. typhi challenge.

The results of the experiments outlined in Examples 1 to 4 indicate that PapMV has intrinsic adjuvant properties that can induce the switch of antigen-specific immunoglobulins, provide a sustained long lasting antibody response to model antigens, and increase the protective capacity of OmpC or OmpF alone. These data indicate that PapMV and PapMV VLPs potentiate the translation of innate and adaptive immune responses elicited by OmpC porin into protection against S. typhi challenge.

### Example 6

### Purification of HCV Core Proteins

Cloning and Expression of HCV core proteins in E. coli

The first N-terminal 82 and 170 amino acids of the HCV core protein (designated as HCV-C82 and HCV-C170, respectively) were optimized with the most representative codons for translation in E. coli and fused to a His6-tag at the C-terminus for purification as follows.

The plasmid pCV-H77c (generously provided by J. Bukh, NIH) was used to generate the HCV Core constructs. HCV-C170 was amplified by PCR with primers C 170-6 h (5′-CATGGGATCCTTACTAATGGTGATGGTGATGGTGACGCGTGGTACTAGTA GGAAGGTTCCCTGTTGCATAGTTCACGCC-3′ [SEQ ID NO:43]), together with primer C N9 (5′-CATGAACCATGGCGAGCACGAATCCTAAACCTCAAAGAAAAACC-3′ [SEQ ID NO:44]). PCR products were digested with restriction enzymes NcoI and BamHI and cloned into a pET3d expression vector (New England Biolabs). Core C-terminal deletion construct HCV-C82 was generated by PCR using the C 1-170 clone as template DNA and the primers 82 (5′-CATGACTAGTAGGGTACCCGGGCTGAGC-3′ [SEQ ID NO:45]), C 79 (5′-ACGTACTAGTGGGCTGAGCCCAGGTCCTGCC-3′ [SEQ ID NO:46]) together with primer c9-6h-Pet (5′-CATGACTAGTACCACGCGTCACC-3′ [SEQ ID NO:47]). The clones were circularized by ligation after digestion of the DNA with SpeI. The sequences of the HCV clones were confirmed by DNA sequencing. The E. coli expression strain BL21(DE3) RIL (Stratagene) was transformed with C protein-expressing pET3d constructs and maintained in 26 YT medium [16 g bacto-peptone 121 (Difco), 10 g yeast extract 121 (Difco), 5 g NaCl 121, adjusted to pH 7.0], supplemented with 50 mg ampicillin m121. Bacterial cells were grown at 37° C. to an OD600 of 0.6 and protein expression was induced with 1 mM IPTG. Induction was continued for 2 h at 25° C. (see Majeau, N., et al. (2004) J. Gen. Virol., 85; 971-981). Cells were pelleted by centrifugation at 6000 g for 15 min. after 2 hours of induction.

### Purification of HCV-C82

The harvested cells were resuspended in a 30 ml of ice cold lysis buffer (50 mM phosphate, 300 mM NaCl, pH 12.0) supplemented with 1× cocktail of protease inhibitors (Roche Diagnostics GmbH) and frozen at −80° C. The cells were then lysed by 24 cycles of 10 sec. sonication followed by 50 sec of cooling between each sonication, using a sonic dismembranator model 500 (Fisher). The lysate was then centrifuged at 27,000 g for 30 min. Supernatant was added to Ni-NTA resin (QIAGEN) with agitation at 4° C. After 90 min. of binding, the beads were washed with 50 ml of washing buffer 1 (50 mM phosphate, 300 mM NaCl, 10 mM imidazole, pH 12.0) and agitated for 30 min. The beads were washed again with 50 ml of washing buffer 2 (50 mM phosphate, 500 mM NaCl, 20 mM imidazole, pH 12.0) and agitated for another 30 min. HCV-C82 was then eluted in assembly buffer 1.7 mM Mg-acetate, 100 mM K-acetate, 25 mM HEPES, pH 7.6, 500 mM imidazole. All steps were carried out at 4° C.

### Purification of HCV-C170

The harvested cells were resuspended in a 30 ml lysis buffer (20 mM Tris/HCl pH (7.4), 8M urea, 300 mM NaCl, 1 mM DTT) supplemented with 500 μm PMSF and frozen at −80° C. The cells were then lysed by 24 cycles of 10 sec. sonication followed by 50 sec of cooling between each sonication, using a sonic dismembranator model 500 (Fisher). The lysate was then centrifuged at 27,000 g for 30 min. Supernatant was added to Ni-NTA resin (QIAGEN) with agitation. After 90 min. of binding, the beads were washed with 50 ml of washing buffer 1 (20 mM Tris/HCl pH (7.4), 4 M urea, 300 mM NaCl 10 mM imidazole, 1 mM DTT) and agitated for 30 min. The beads were washed again with 50 ml of washing buffer 2 (20 mM Tris/HCl pH (7.4), 2 M urea, 300 mM NaCl, 20 mM imidazole, 1 mM DTT) and agitated for another 30 min. HCV-C170 was then eluted in elution buffer (20 mM Tris/HCl pH (7.4), 2M urea, 500 mM NaCl, 500 mM imidazole, 1 mM DTT). All these steps were carried out at room temperature.

### Reverse-Phase HPLC Purification of HC V-Core Protein

Reversed-phase HPLC was carried out on a HP 1050 series (Hewlett Packard) HPLC with a UV detector and a VYDAC C4 column (250 mm×4.6 mm, 5 μm, and 300 A). The solvents used for the gradient were 0.05% trifluoroacetic acid in water (solvent A) and 0.05% trifluoroacetic acid in acetonitrile (solvent B). The flow rate was 0.8 ml/min with solvent B increasing from 10% to 50% in 35 min for HCV-C82 and from 10% to 50% in 15 min. and to 60% in 20 min. for HCV-C170. The chromatograms were recorded at 220 nm and 280 nm and data analyzed using ChemStation for LC 3D (Agilent Technologies). The collected fractions were lyophilised and reconstituted in PBS for further studies.

### Protein Characterization

Protein purity and quantity were estimated by SDS-polyacrylamide gel electrophoresis (SDS-PAGE) and the Bicinchoninic acid (BCA) protein assay kit (Pierce).

HCV-C82 and HCV-C170 assembly into NLPs

In vitro assembly reactions were carried out using the purified HCV C proteins and tRNA (Sigma). Purified protein (25 mg in 50 ml assembly buffer; (1 mM magnesium acetate, 100 mM potassium acetate, 25 mM HEPES, pH 7.4)) was mixed with 250 ng tRNA in the presence of 16 protease inhibitor cocktail and 0.5 U RNase inhibitor (Roche). The reactions were incubated at 37° C. for 10 min followed by 15 min on ice (see Majeau, N., et al. (2004) J. Gen. Virol., 85; 971-981.)

### Example 7

### Production and Engineering of PapMV VLPs Comprising HCV Core Affinity Peptides

### Phage Display

A Ph.D.-7 phage library supplied by New England Biolabs (Beverly, USA) was used. The random displayed heptapeptides used in this library are fused at the N-terminus of PIII protein of M13 phage. The library consists of 70 copies of each 1.28×10⁹ of 7 residues possible in 10 μl of the supplied phage. HCV-C170 was used as the protein target for the panning procedure and was diluted to 100 μg/ml in 0.1 M of NaHCO₃ (pH 8.6) and 150 μl was adsorbed to one well of a maxisorp 96-well polystyrene plate (Nunc, Roskilde, Denmark). The plate was incubated at 4° C. overnight with gentle agitation in a humidified container. Non adsorbed protein was poured off and the well was blocked by adding 4001 of blocking buffer (0.1 M NaHCO₃ (pH 8.6)+5 mg/ml BSA+0.02% (w/v) NaN₃) and incubating the plate 1 h at 4° C. Each well was washed six times with Tris buffered saline (TBS): 50 mM Tris-HCl, 150 mM NaCl (pH 7.5) supplemented with 0.1% (v/v) Tween-20. Ten μl of phage library Ph.D.-7 (2×10¹¹ pfu) was diluted to 1 ml in TBST and 100 μl was dispensed in each well. The plate was incubated for 1 h at room temperature with gentle agitation. Unbound phage were removed from the wells by washing ten times with TBST. Bound phage were eluted by 100 μl of target protein at 100 μg/ml in TBS. This panning procedure was repeated for a total of four rounds in two independent experiments. For each subsequent round of panning the input number of phage was 2×10¹¹ pfu. Stringency of each selection was increased by using 0.5% Tween-20 in TBS for the three last rounds of panning to reduce the frequency of nonspecific phage binding.

### Phage Titration

A single colony of E. coli ER2738 was inoculated in 10 ml of LB and incubated with shaking until mid-log phase (OD₆ₒₒ˜0.5). A 10-fold serial dilution of eluted phage were prepared in LB, in a range of 10⁸-10¹¹ for amplified phages or 10¹-10⁴ for crude panning eluate. 10 μl of each dilution was added to 200 μl of mid-log phase bacteria and incubated at room temperature for 5 min. Infected cells were transferred to a culture tube containing pre-warmed agarose top (45° C.), vortexes quickly, and poured onto a prewarmed LB/IPTG/Xgal plates. Plates were incubated overnight at 37° C. and plates containing ˜100 lysis plaques were counted for titration.

### Plaque Amplification

Overnight cultures of E. coli ER2738 were diluted 1:100 in LB and inoculated with a blue plaque selected from plates having 10 to ˜100 plaques. Inoculated tubes were incubated at 37° C. with shaking for 4-5 hours. After incubation, cultures were centrifuged 30 seconds and supernatants were transferred to a fresh tube and re-spun. Using a pipet, the upper 80% of the supernatants was transferred to a clean tube and amplified phage were stored at 4° C. until the next processing.

### Phage Sequencing

For DNA extraction, QIAprep® spin M13 kit (Qiagen) was used following the manufacturer's instructions. 10 clones of the final panning procedures for each experiment were DNA sequenced using the following primer: 5′TGTATGGGATTTTGTAATACATCA 3′[SEQ ID NO:37].

### Cloning of PapMV Coat Protein and Generation of PapMVCP-STASYTR

The PapMV CP gene was amplified by RT/PCR from isolated viral RNA using the primers: 5′-AGTCCCATGGCATCCACACCCAACATAGCCTTC-3′ [SEQ ID NO:38] and 5′-GATCGGATCCTTACTAATGGTGATGGTGATGGTGACGCGTGGTACTAGTTT CGGGGGGTGGAAGGAATTGGATGGTTGG-3′ [SEQ ID NO:39]. The amplified fragment was cloned as a NcoI/BamHI fragment into pET 3D (New England Biolabs).

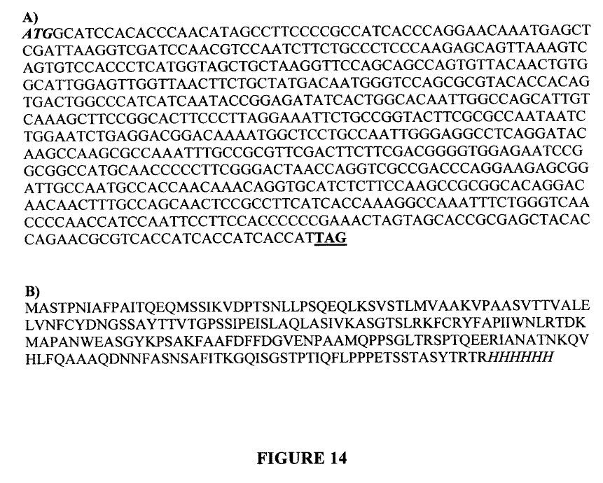

To generate the PapMVCP-STASYTR construct ([SEQ ID NO:42]; see FIG. 14) the oligos 5′CTAGTAGCACCGCGAGCTACACCAGAA-3′ [SEQ ID NO:40] and 5′ CGCGTTCTGGTGTAGCTCGCGGTGCTA-3′ [SEQ ID NO:41], were annealed together and ligated into the PapMV CP clone linearized with SpeI/MIul. The nucleic acid sequence of the final PapMVCP-STASYTR construct is shown in FIG. 14A [SEQ ID NO:48].

### PapMVCP Protein Production and Purification

The E. coli expression strain BL21 (DE3) RIL (Stratagene) was transformed with the plasmid pET-3d containing PapMVCP-STASYTR. Cultures were grown with 50 μg/ml ampicillin at 37° C. to an OD₆ₒₒ of approximately 0.6, induced at 22° C. overnight using 1 mM IPTG (isopropyl-1-thio-β-D-galactopyranoside), and centrifuged at 6000 g for 15 min. The pellet was resuspended in ice-cold lysis buffer (50 mM Na H₂PO₄ [pH 8.0], 300 mM NaCl, 10 mM imidazole, 40 μM PMSF, and 0.1 mg/ml lysozyme) and bacteria were lysed by one passage through a French Press at 750 PSI. The lysate was centrifuged twice for 30 min at 13,000 rpm to eliminate cellular debris. The whole purification procedure was performed at 4° C. The supernatant was mixed overnight with 1 ml of Ni-NTA-agarose matrix (QiagenD40724). The protein was purified by gravity-flow on a polypropylene chromatography column (Econo-Pac Columns, Bio-Rad Laboratories). The resin was washed with 10 bed volumes once with a first wash buffer (lysis buffer supplemented with 20 mM imidazole) and once with the second wash buffer (lysis buffer supplemented with 50 mM imidazole). In order to remove LPS contaminants from the preparations, they were washed once with 10 mM Tris-HCl 50 mM imidazole 0.5% Triton X100 pH 8 and once with 10 mM Tris-HCl 50 mM imidazole 1% Zwittergent pH 8. Following these washing steps, a wash with a third wash buffer (10 mM Tris-HCl (pH 8.0) and 50 mM imidazole) was performed to remove all traces of detergent. The protein was incubated overnight with 2.5 bed volumes of the elution buffer (third wash buffer supplemented with 1M imidazole) and dialysed overnight in 10 mM Tris-HCl (pH 8.0). To increase the amount of VLPs in the sample, the sample was centrifuged at 3,000×g for 90 min. through a 1000 k Macrosep® centrifugal devices (Pall life sciences) and dialysed overnight in PBS.

### Protein Characterisation

Endotoxin content of vaccinal preparations was estimated with a Limulus amoebocyte lysate assay kit (Cambrex). VLP content was evaluated by FPLC size-exclusion chromatography using a Superdex 200 (10/300) GL analytical column.

### Electron Microscopy

After assembly of the HCV core proteins into NLPs, 150 ng of the sample was diluted in PBS and adsorbed onto 400-mesh carbon formvar grids (Canemco) for 5 min. Grids were washed once with TBS and stained for 3 min. with filtered 2% uranyl acetate solution. Grids were the dried and examined under an electronic microscope with an accelerated voltage of 60 kVol at a magnification of 100,000.

### ELISA

Costar High Binding 96-well plates (Corning, N.Y., U.S.A.) were coated overnight at 4° C. with 100 μl/well of HCV-C170 NLP or free (i.e. non-NLP) HCV-C170 diluted to a concentration of 1 μg/ml in 0.1 M NaHCO₃ buffer pH 9.6. The plates were blocked with PBS-0.1% Tween-20-2% BSA (150 μl/well) for 1 hour at 37° C. 2-fold serial dilution of PapMVCP-STASYTR was tested beginning at 1 μg/ml or 2.5 μg/ml. The plates were incubated with 100 μl of polyclonal rabbit antibody raised against PapMVCP protein at 1/5000 in PBST before incubation with peroxidase-conjugated goat anti-rabbit IgG. After three washes, the presence of IgG was detected with 100 μl of TMB-S according to the manufacturer's instructions; the reaction was stopped by adding 100 μl of 0.18 mM H₂SO₄ and the OD was read at 450 nm.

### Results

Affinity peptides capable of binding to HCV core protein were selected by phage display. The sequences of the peptides and their frequency are shown in Table 5.

TABLE 5
Sequence and Frequency of Occurrence of HCV
Core Affinity Peptides

| Sequence of Peptide | Frequency | SEQ ID NO |
|---|---|---|
| STASYTR | 4/10 | 8 |
| NASSLRS | 1/10 | 15 |
| HSPKNLH | 1/10 | 16 |
| NTPQGMT | 1/10 | 17 |
| GPSTPIR | 1/10 | 18 |
| GVQIMGR | 1/10 | 19 |
| SIQYTGV | 1/10 | 20 |

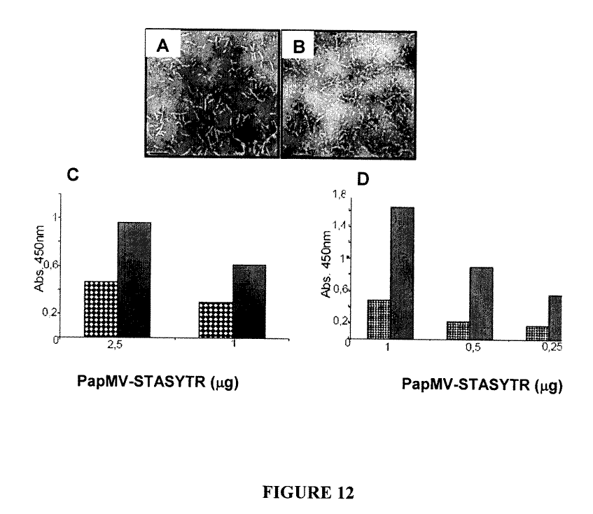

The peptide STASYTR [SEQ ID NO:8] was found with the highest frequency and was therefore chosen to be fused to the PapMV coat protein. Electron microscopy (EM) observations confirmed that the addition of the peptides to the PapMV CP did not affect the ability of the resulting fusion protein, PapMVCP-STASYTR, to self-assemble into VLPs (FIG. 12B) as compared to the PapMV CP without the fusion (FIG. 12A).

The binding of PapMVCP-STASYTR with a HCV core protein (1-170) NLP and free (non-NLP) HCV-C170 was shown by an ELISA-type binding assay. For both assays, binding to the antigen was clearly demonstrated and increased with the amount of the PapMVCP-STASYTR used in the assay (FIGS. 12C and D).

### Example 8

### Immunization Against Hepatitis C Virus with an Affinity-Conjugated PapMV VLP-HCV Core

### Preparation of PapMV-STASYTR/HCV-Core ACAS

PapMV-STASYTR and HCV-C82 were mixed together in sterile PBS buffer in a ratio of 5:1 PapMV-STASYTR: HCV-C82 and then incubated for 16 hours at 4° C. Following this first incubation, the mixture was submitted to a second incubation for 90 min at 25° C. (room temperature).

### Mice

Five 4-8 weeks old C57BL/6 mice were injected subcutaneously with 10 μg of HCV-C82 NLPs or 10 μg of C82 NLPs+50 μg PapMVCP-STASYTR or 50 μg PapMVCP-STASYTR or PBS endotoxin-free (Sigma).

Primary immunization was followed by 2 booster doses given at 2 week intervals. Blood samples were obtained at different time points and stored at −20° C. until analyzed. Three weeks after the last immunization, mice were challenged intraperitoneally with 5×10⁶ PFU of recombinant vaccinia virus Sc59 6C/Ss expressing amino acids 1-382 of the HCV polyprotein. (Koziel et al., 1995 J Clin Invest. 96(5):2311-21). Mice were anesthetized before challenge with 0.1 ml of ketamin (15 mg/ml)-Xylazin (1 mg/ml) per 10 g body weight. Mice were sacrificed 5 days after the challenge and ovaries were removed, homogenized, and assayed for viral titer by serial 10-fold dilutions on a plate of CV-1 indicator cells. After 2 days of culture, the medium was removed, the CV-1 cell monolayer was stained with 1% methylene blue (Sigma) for 10 min, washed 10 min. with water and the number of plaques per well was counted for titration of vaccinia virus.

### ELISA

Costar High Binding 96-well plates (Corning, N.Y., U.S.A.) were coated overnight at 4° C. with 100 μl/well of HCV-C170 free protein diluted to a concentration of 1 μg/ml in 0.1 M NaHCO₃ buffer pH 9.6. The plates were blocked with PBS-0.1% Tween-20-2% BSA (150 μl/well) for 1 hour at 37° C. After washing three times with PBS-0.1% Tween-20, sera were tested in 2-fold serial dilution beginning from 1:100 were added and incubated for 1 hour at 37° C. Following incubation, the plates were washed three times and incubated with 100 μl of peroxidase-conjugated goat anti-mouse IgG (Jackson Immunoresarch) at a dilution of 1/10,000 in PBS/0.1% Tween-20/2% BSA for 1 hour at 37° C. After three washes, the presence of IgG was detected with 100 μl of TMB-S according to the manufacturer's instructions; the reaction was stopped by adding 100 μl of 0.18 mM H₂SO₄ and the OD was read at 450 nm. The results are expressed as antibody endpoint titer, determined when the OD value is 3-fold the background value obtained with a 1:100 dilution of serum from PBS mice.

### Statistical Methods

Data were transformed X=(log y+1) to homogenize variance and then analysed using an ANOVA test with a Tukey's multiple comparison as a post test for parametric data. P<0.05 was considered statistically significant.

### Results

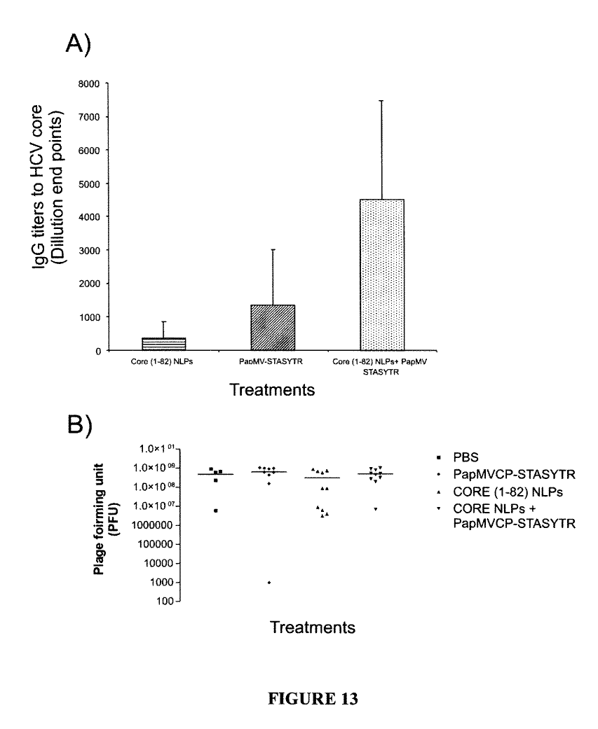

Total IgG titers in mice following immunization with HCV core, PapMVCP-STASYTR, or HCV core+PapMVCP-STASYTR are shown in FIG. 13A. Immunization with HCV core+PapMVCP-STASYTR shows a statistically significant (p<0.05) increase in IgG compared to immunization with HCV core alone or PapMVCP-STASYTR alone.

FIG. 13B shows viral titers recovered from both ovaries of infected mice following challenge with recombinant vaccinia virus Sc59 6C/Ss expressing amino acids 1-382 of the HCV polyprotein. Although in this experiment, no significant difference was observed following immunization with PBS, HCV core, PapMVCP-STASYTR or HCV core+PapMVCP-STASYTR, it is clear from the total IgG titers that immunization with HCV core+PapMVCP-STASYTR composition is capable of producing a superior immune response to that achieved with HCV core alone. As such, it is predicted that optimization of the composition and/or administration routine by, for example, addition of a booster administration of antigen alone, will result in effective protection against viral challenge. Such optimization can be readily undertaken by the skilled worker in light of the teaching provided herein. For example, CD8+ HCV core specific proliferation assays are being performed to determine the optimal ratio of PapMV-STASYTR and HCV core protein that is able to trigger a potent CTL response in mice, as well as appropriate doses of the final vaccine preparation. In one embodiment, ratios of 4:1, 3:1, 2:1 and 1:1 of PapMV-STASYTR to HCV core protein are contemplated for preparation of the ACAS for inclusion in vaccine preparations.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. An affinity-conjugated antigen system comprising one or more antigens and a papaya mosaic virus (PapMV) or a virus-like particle (VLP) derived from PapMV coat protein, said PapMV or VLP comprising a plurality of affinity moieties attached to coat proteins of the PapMV or VLP, said affinity moieties capable of binding said one or more antigens, wherein said system is capable of inducing an immune response in an animal.

2. The affinity-conjugated antigen system of claim 1, wherein said one or more affinity moieties are chemically attached to the coat protein of the PapMV or VLP.

3. The affinity-conjugated antigen system of claim 1, wherein said system comprises a VLP and said one or more affinity moieties are genetically fused to the coat protein of the VLP.

4. The affinity-conjugated antigen system of claim 1, wherein said one or more affinity moieties are peptides.

5. The affinity-conjugated antigen of claim 1, wherein said one or more antigens are each a tumour-associated antigen, self-antigen, allergen, viral antigen, bacterial antigen or parasitic antigen.

6. The affinity-conjugated antigen system of claim 1, wherein said one or more antigens are each derived from a bacterium, virus, protozoan, fungus, parasite, or infectious particle.

7. The affinity-conjugated antigen system of claim 1, wherein said one or more antigens are derived from a virus.

8. The affinity-conjugated antigen system of claim 1, wherein said one or more antigens are derived from a bacterium.

9. The affinity-conjugated antigen system of claim 1, wherein said immune response comprises a humoral response.

10. The affinity-conjugated antigen system of claim 1, wherein said immune response comprises a cellular response.

11. The affinity-conjugated antigen system of claim 1, wherein said system further comprises one or more additional antigens.

12. An immunogenic composition comprising the affinity-conjugated antigen system according to claim 1, and a pharmaceutically acceptable carrier.

13. A method of inducing an immune response in an animal comprising administering to said animal an effective amount of the affinity-conjugated antigen system according to claim 1.

14. The method according to claim 13, wherein said immune response comprises the production of antibodies.

15. The method according to claim 13, wherein said immune response comprises the induction of a cytotoxic T lymphocyte (CTL) response.

16. The method according to claim 13, wherein said affinity-conjugated antigen system is administered by injection.

17. The method according to claim 13, wherein said affinity-conjugated antigen system is administered intranasally.

18. The method according to claim 13, wherein said animal is a mammal.

19. The method according to claim 13, wherein said animal is a human.

20. The method according to claim 13, wherein said animal is a non-human animal.

21. The method according to claim 13, wherein said method further comprises administering to said animal a booster dose of said one or more antigens.

22. A method of preventing or treating a disease or disorder in an animal, said method comprising administering to said animal an effective amount of the antigen presenting system according to claim 1.

23. The method according to claim 22, wherein induction of a humoral immune response is effective to prevent or treat said disease or disorder.

24. The method according to claim 22, wherein said disease or disorder is caused by a bacterium.

25. The method according to claim 22, wherein said disease or disorder is caused by a virus.

26. The method according to claim 22, wherein said affinity-conjugated antigen system is administered by injection.

27. The method according to claim 22, wherein said affinity-conjugated antigen system is administered intranasally.

28. The method according to claim 22, wherein said animal is a mammal.

29. The method according to claim 22, wherein said animal is a human.

30. The method according to claim 22, wherein said animal is a non-human animal.

31. The method according to claim 22, wherein said method further comprises administering to said animal a booster dose of said one or more antigens.

32-55. (canceled)

56. A method of preparing an immunogenic composition comprising admixing one or more antigens with a papaya mosaic virus (PapMV) or a virus-like particle (VLP) derived from PapMV coat protein, said PapMV or VLP comprising a plurality of affinity moieties attached to coat proteins of said PapMV or VLP, said affinity moieties capable of binding said one or more antigens.

57. An immunogenic composition prepared by the method according to claim 56.

58. A fusion protein comprising a papaya mosaic virus (PapMV) coat protein fused to an affinity peptide capable of binding to HCV core protein.

59. The fusion protein according to claim 58, wherein said affinity peptide comprises at least four consecutive amino acids of the sequence as set forth in any one of SEQ ID NOs: 8, 15, 16, 17, 18, 19 or 20.

60. An isolated polynucleotide encoding the fusion protein according to claim 58.

61. (canceled)

62. A virus-like particle comprising the fusion protein according to claim 58.

63. The method according to claim 12, wherein said one or more affinity moieties are chemically attached to the coat protein of the PapMV or VLP.

64. The method according to claim 12, wherein said system comprises a VLP and said one or more affinity moieties are genetically fused to the coat protein of the VLP.

65. The method according to claim 12, wherein said one or more affinity moieties are peptides.

66. The method according to claim 12, wherein said one or more antigens are each a tumour-associated antigen, self-antigen, allergen, viral antigen, bacterial antigen or parasitic antigen.

67. The method according to claim 12, wherein said one or more antigens are each derived from a bacterium, virus, protozoan, fungus, parasite, or infectious particle.

68. The method according to claim 12, wherein said one or more antigens are derived from a virus.

69. The method according to claim 12, wherein said one or more antigens are derived from a bacterium.

70. The method according to claim 12, wherein said system further comprises one or more additional antigens.

71. The method according to claim 22, wherein said one or more affinity moieties are chemically attached to the coat protein of the PapMV or VLP.

72. The method according to claim 22, wherein said system comprises a VLP and said one or more affinity moieties are genetically fused to the coat protein of the VLP.

73. The method according to claim 22, wherein said one or more affinity moieties are peptides.

74. The method according to claim 22, wherein said one or more antigens are each a tumour-associated antigen, self-antigen, allergen, viral antigen, bacterial antigen or parasitic antigen.

75. The method according to claim 22, wherein said one or more antigens are each derived from a bacterium, virus, protozoan, fungus, parasite, or infectious particle.

76. The method according to claim 22, wherein said one or more antigens are derived from a virus.

77. The method according to claim 22, wherein said one or more antigens are derived from a bacterium.

78. The method according to claim 22, wherein said system further comprises one or more additional antigens.


SEQUENCE LISTING (48 sequences)

SEQ ID NO: 1 - protein, 215 aa, Papaya mosaic virus
  wild-type coat protein
MSKSSMSTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLIRSPTQEERIANATNKQVHLFQAAAQDNNFTSNSAFITKGQISGSTPTIQFLPPPE

SEQ ID NO: 2 - DNA, 648 bp, Papaya mosaic virus
atgtctaagtcaagtatgtccacacccaacatagccttccccgccatcacccaggaacagatgagctcgattaaggtcgatccaacgtccaatcttctgccctcccaagagcagttaaagtcagtgtccaccctcatggtagctgctaaggttccagcagccagtgttacaactgtggcattggagttggtcaacttctgctatgacaatgggtccagcgcgtacaccacagtgactggcccatcatcaataccggagatatcactggcacaattggctagtattgtcaaagcttccggcacttcccttagaaaattctgccggtacttcgcgccaataatctggaatctgaggacggacaaaatggctcctgccaattgggaggcttcaggatacaagccaagcgccaaatttgccgcgttcgacttcttcgacggggtggagaatccggcggccatgcaacccccttcgggactaatcaggtcgccgacccaggaagagcggattgccaatgctaccaacaaacaggtgcatctcttccaagccgcggcacaggacaacaactttaccagcaactccgccttcatcaccaaaggccaaatttctgggtcaaccccaaccatccaattccttccaccccccgaataa

SEQ ID NO: 3 - protein, 211 aa, Artificial Sequence
  mutant coat protein CPdelta5
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPE

SEQ ID NO: 4 - protein, 318 aa, Salmonella enterica subsp. enterica serovar Typhi
  OmpC precursor protein
MKVKVLSLLVPALLVAGAANAAEIYNKDGNKLDLFGKVDGLHYFSDDKGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGFSVGGAITTSKRTADQNNTANARLYGNGDRATVYTGGLKYDANNIYLAAQYSQTYNATRFGTSNGSNPSTSYGFANKAQNFEVVAQYQFDFGLRDAGINTDDIVALGLVYQF

SEQ ID NO: 5 - protein, 363 aa, Salmonella enterica subsp. enterica serovar Typhi
  OmpF precursor protein
MMKRKILAAVIPALLAAATANAAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGADGSADLAKYIQAGATYYFNKNMNVWVDYRFNLLDENDYSSSYVGTDDQAAVGITYQF

SEQ ID NO: 6 - protein, 224 aa, Artificial Sequence
  PapMV coat protein comprising an affinity peptide for binding to OmpC
MASTPNIAFPAITQEQMSSIEVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQGNNFASNSAFITKGQISGSTPTIQFLPPPEEAKGLIRHHHHHH

SEQ ID NO: 7 - protein, 224 aa, Artificial Sequence
  PapMV coat protein comprising an affinity peptide for binding to OmpF
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPEFHENWPSHHHHHH

SEQ ID NO: 8 - protein, 7 aa, Artificial Sequence
  HCV Core Affinity peptide
STASYTR

SEQ ID NO: 9 - protein, 7 aa, Artificial Sequence
  S. typhi OmpC Affinity peptide
SLSLIQT

SEQ ID NO: 10 - protein, 7 aa, Artificial Sequence
  S. typhi OmpC Affinity peptide
EAKGLIR

SEQ ID NO: 11 - protein, 7 aa, Artificial Sequence
  S. typhi OmpC Affinity peptide
TATYLLD

SEQ ID NO: 12 - protein, 7 aa, Artificial Sequence
  S. typhi OmpF Affinity peptide
FHENWPS

SEQ ID NO: 13 - protein, 7 aa, Artificial Sequence
  S. typhi OmpF Affinity peptide
FHEFWPT

SEQ ID NO: 14 - protein, 7 aa, Artificial Sequence
  S. typhi OmpF Affinity peptide
FHEXWPT

SEQ ID NO: 15 - protein, 7 aa, Artificial Sequence
  HCV Core Affinity peptide
NASSLRS

SEQ ID NO: 16 - protein, 7 aa, Artificial Sequence
  HCV Core Affinity peptide
HSPKNLH

SEQ ID NO: 17 - protein, 7 aa, Artificial Sequence
  HCV Core Affinity peptide
NTPQGMT

SEQ ID NO: 18 - protein, 7 aa, Artificial Sequence
  HCV Core Affinity peptide
GPSTPIR

SEQ ID NO: 19 - protein, 7 aa, Artificial Sequence
  HCV Core Affinity peptide
GVQIMGR

SEQ ID NO: 20 - protein, 7 aa, Artificial Sequence
  HCV Core Affinity peptide
SIQYTGV

SEQ ID NO: 21 - protein, 23 aa, Artificial Sequence
  Influenza virus M2e peptide
SLLTEVETPIRNEWGCRCNDSSD

SEQ ID NO: 22 - protein, 23 aa, Artificial Sequence
  Influenza virus M2e peptide
SLLTEVETPIRNEWGCRCNGSSD

SEQ ID NO: 23 - protein, 23 aa, Artificial Sequence
  Influenza virus M2e peptide
SLLTEVETPTKNEWDCRCNDSSD

SEQ ID NO: 24 - protein, 23 aa, Artificial Sequence
  Influenza virus M2e peptide
SLLTEVETPTRNGWECKCSDSSD

SEQ ID NO: 25 - protein, 23 aa, Artificial Sequence
  Influenza virus M2e peptide
SLLTEVETPTRNEWECRCSDSSD

SEQ ID NO: 26 - protein, 8 aa, Artificial Sequence
  Influenza virus M2e peptide
EVETPIRN

SEQ ID NO: 27 - protein, 8 aa, Artificial Sequence
  Influenza virus M2e peptide
EVETLTRN

SEQ ID NO: 28 - protein, 8 aa, Artificial Sequence
  Influenza virus M2e peptide
EVETPIRS

SEQ ID NO: 29 - protein, 8 aa, Artificial Sequence
  Influenza virus M2e peptide
EVETPTRN

SEQ ID NO: 30 - protein, 8 aa, Artificial Sequence
  Influenza virus M2e peptide
EVETPTKN

SEQ ID NO: 31 - protein, 8 aa, Artificial Sequence
  Influenza virus M2e peptide
EVDTLTRN

SEQ ID NO: 32 - protein, 8 aa, Artificial Sequence
  Influenza virus M2e peptide
EVETPIRK

SEQ ID NO: 33 - protein, 8 aa, Artificial Sequence
  Influenza virus M2e peptide
EVETLTKN

SEQ ID NO: 34 - DNA, 45 bp, Artificial Sequence
  PapN-terminus (Forward)  Primer
atcgccatggcatccacacccaacatagccttccccgccatcacc

SEQ ID NO: 35 - DNA, 76 bp, Artificial Sequence
  PapOmpC (Reverse) Primer
ggttaaggaaggtggggggcttctccgcttccccaactaagcatggtagtggtagtggtaatcattcctaggtgac

SEQ ID NO: 36 - DNA, 77 bp, Artificial Sequence
  PapOmpF (Reverse) Primer
ggttaaggaaggtggggggcttaaagtactcttaaccggaagcgtggtagtggtagtggtaatcattcctaggtgac

SEQ ID NO: 37 - DNA, 24 bp, Artificial Sequence
  Primer
tgtatgggattttgtaatacatca

SEQ ID NO: 38 - DNA, 33 bp, Artificial Sequence
  Primer
agtcccatggcatccacacccaacatagccttc

SEQ ID NO: 39 - DNA, 79 bp, Artificial Sequence
  Primer
gatcggatccttactaatggtgatggtgatggtgacgcgtggtactagtttcggggggtggaaggaattggatggttgg

SEQ ID NO: 40 - DNA, 27 bp, Artificial Sequence
  Oligonucleotide
ctagtagcaccgcgagctacaccagaa

SEQ ID NO: 41 - DNA, 27 bp, Artificial Sequence
  Oligonucleotide
cgcgttctggtgtagctcgcggtgcta

SEQ ID NO: 42 - protein, 228 aa, Artificial Sequence
  PapMV coat protein comprising an affinity peptide for binding to HCV core protein fragment
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSSTASYTRTRHHHHHH

SEQ ID NO: 43 - DNA, 79 bp, Artificial Sequence
  Primer
catgggatccttactaatggtgatggtgatggtgacgcgtggtactagtaggaaggttccctgttgcatagttcacgcc

SEQ ID NO: 44 - DNA, 44 bp, Artificial Sequence
  Primer
catgaaccatggcgagcacgaatcctaaacctcaaagaaaaacc

SEQ ID NO: 45 - DNA, 28 bp, Artificial Sequence
  Primer
catgactagtagggtacccgggctgagc

SEQ ID NO: 46 - DNA, 31 bp, Artificial Sequence
  Primer
acgtactagtgggctgagcccaggtcctgcc

SEQ ID NO: 47 - DNA, 23 bp, Artificial Sequence
  Primer
catgactagtaccacgcgtcacc

SEQ ID NO: 48 - DNA, 687 bp, Artificial Sequence
  Nucleotide sequence encoding PapMV coat protein comprising an affinity peptide for binding to HCV core protein fragment
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtagcaccgcgagctacaccagaacgcgtcaccatcaccatcaccattag